(19) United States
(12) Patent Application Publication
(10) Pub. No.: US 2026/0034169 A1
(43) Pub. Date: Feb. 5, 2026

(54) STABILISED SILICIC ACID SOLUTIONS

(71) Applicant: BARLAA B.V., Muiden (NL)

(72) Inventor: Ivica CEPANEC, Kriz (HR)

(73) Assignee: BARLAA B.V., Muiden (NL)

(21) Appl. No.: 19/099,504

(22) PCT Filed: Jul. 6, 2023
(86) PCT No.: PCT/EP2023/068761

§ 371 Date: Jan. 29, 2025

(30) Foreign Application Priority Data
Aug. 2, 2022 (EP) 22188329.1

(51) Int. Cl. A61K 33/00; A61K 8/25; A61K 9/08; A61K 47/02; A61K 47/10; A61K 47/18; A61Q 19/00

(52) U.S. Cl. CPC A61K 33/00; A61K 8/25; A61K 9/08; A61K 47/02; A61K 47/10; A61K 47/183; A61Q 19/00

(57) ABSTRACT

The present invention relates to stabilized silicic acid solutions, with 0.01-1.00% w/w silicon content. Silicic acid is stabilized by the combination of amino acid hydrogensulfate 1AA, 1b, 1c, sodium and/or potassium amino acid sulfate 2aAA, 2bAA, 2c, 2d, 2e, 2f, and sodium and/or potassium hydrogensulfate (NaHSO₄ and/or KHSO₄) in a mixture of a diol, 1,2-propylene glycol (3) and/or polyethylene glycol (4), and purified water. The invention discloses the process for the preparation of this formulation. The composition is used as a food supplement, functional food ingredient, feed additive, functional feed additive, biostimulant for aquaculture, active cosmetic ingredient, active pharmaceutical ingredient, as an agrochemical product, etc., that provides a highly bioavailable silicon (Si).

### FIELD OF THE INVENTION

The present invention relates to compositions comprising silicic acid, which may, e.g., be used as a pharmaceutical, nutraceutical, cosmetic, veterinary, or agrochemical product providing beneficial effects of silicon (Si) on eukaryotes, including humans, animals, diatoms, and plants. The present invention, in particular, addresses the technical problem of stabilizing silicic acid against its polymerisation in solution.

### BACKGROUND OF THE INVENTION

Silicon (Si) is an important micronutrient that exhibits numerous beneficial effects in humans, animals, and plants.

It is known that the main bioavailable chemical form of silicon is ortho-silicic (also called monosilicic) acid (OSA). However, the latter is prone to the polymerisation into its oligomers, various types of polymers, and finally to silicic acid gel (SG), see FIG. 1.

The main chemical form of silicon is ortho-silicic acid (H₄SiO₄; OSA) [1,2]. A saturated solution contains 0.1% w/w silicic acid [2].

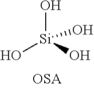

In more concentrated solutions, OSA undergoes a polymerisation process which is accompanied by the elimination of water (H₂O) and the formation of different oligomers and polymers such as:
(i) oligomers, e.g., of type HO—[Si(OH)₂O]ₙ—H with n≤12 and molar mass, M<700 [g/mol] (water-soluble):
   (a) linear oligomers, e.g., dimeric silicic acid (S2), trimeric silicic acid (S3);
   (b) branched oligomers, e.g., branched tetrameric silicic acid (bS4);
   (c) cyclic oligomers, e.g., cyclic trimer (cS3), cyclic tetramer (cS4);
   (d) cage-type sesquisiloxanes like compound SS;
(ii) polymers of general formula [SiOₓ(OH)₄₋₂ₓ]ₙ, x=1-2, as follows:
   (e) sub-nano silicic acid (SNSA) of particles size 0.3-0.6 nm, n=12-20 and M=700-1,400 [g/mol]; which is freely water-soluble;
   (f) sub-nano condensed silicic acid (SNCSA) of particles size 0.6-3 nm, n=20-300 and M=1,400-2,000 [g/mol]; which is freely water-soluble;
   (g) larger SNCSA silicic acid of particles size 3-5 nm, n=300-2,000 and M=20,000-140,000 [g/mol]; which is still freely or moderately water-soluble;
   (h) polymerised silica nano-particles (PSNP) of particles size 5-50 nm, n=2,000-10,000 and M=140,000-600,000 [g/mol]; which finally ends in,
(iii) highly polymeric silicic acid [(SiO₂)ₙ·yH₂O; silica-gel; SG] of particles size>50 nm, n>10,000 and M=>600,000 [g/mol].

Besides OSA itself, its water-soluble oligomers and lower polymers, which can release a certain amount of free OSA, represent valuable silicon-containing chemical forms that provide said beneficial nutraceutical and pharmacological effects of silicon in humans, and animals and also in plant production and protection.

In contrast, water-insoluble, higher silicic acid polymers and silica gel release very small amounts of ortho-silicic acid and thus are less favourable chemical forms for silicon supplementation or therapeutic use.

A lot of effort has therefore been devoted to the development of stabilized silicic acid solutions, which will not form a gel (a water-insoluble polymer).

In order to prevent, minimise, or slow down the polymerisation of OSA, which is accompanied by gelling, its oligomers, and lower polymers to water-insoluble higher polymers of poor silicon bioavailability, several approaches to the formulation of OSA solutions have been developed and described.

WO 2011/071379 discloses an acidic aqueous solution of micro-colloidal silicic acid, boric acid (H₃BO₃), and a water-absorbing additive having a pH value of equal to or less than 1, wherein at least 90% of the micro-colloidal silicic acid particles have a particles size in the range of 3.5-8 nm. The water-absorbing additive or humectant is selected from the group consisting of polysorbate, vegetable gum, substituted cellulose, polyethylene glycol (PEG), polydextrose, propylene glycol, propylene glycol alginate, polyoxyethylene glycol ester, pectin, amidated pectin, sucrose ester of fatty acid, acetylated or hydroxypropyl starch, starch phosphate, urea, sorbitol, maltitol, vitamin, and a mixture of two or more of such humectants. WO 2011/071379 focuses on the stabilisation of silicic acid nano-particles of size 3.5-8 nm by the influence of boric acid (H₃BO₃). This document does not mention glycine or its salts as a silicic acid stabiliser.

WO2012/035364 discloses a liquid composition of ortho-silicic acid (OSA) stabilized with carnitine salts such as carnitine hydrogenphosphate, within, among others, an aqueous glycerol, 1,2-propylene glycol, d-panthenol or glucosamine matrix. A serious disadvantage of the compositions according to WO 2011/071379 is the use of tetraethyl orthosilicate (TEOS) as a silicon precursor. The use of TEOS is connected with certain safety aspects and the final product inevitably contains four moles of ethanol per each mole of released OSA. The content of alcohol makes this approach less favourable.

EP3141244A1 discloses new compositions of stabilized ortho-silicic acid (OSA) based on its stabilization by its incorporation into a water-in-oil (W-O) emulsion. Such emulsion in its water phase contains stabilizer selected from the group consisting of polysorbate, vegetable gum, cellulose, polyglycerol esters, polyethylene glycol (PEG), dextrose, propylene glycol, and sugars. A serious disadvantage of the compositions according to EP3141244A1 is that it requires the use of a fatty phase and unavoidable emulsifier. The use of emulsifiers and other food additives of significant health concerns is not favourable in modern trends in nutraceutical product formulations.

WO 03/077657 discloses a liquid stabilized silicic acid solution that contains ortho-silicic acid (H₄SiO₄) and its oligomers, that is prepared from silicon tetrachloride (SiCl₄) by hydrolysis with water in the presence of hydrogen chloride (HCl) in 75% aqueous solution of choline chloride (ChCl) in a molar ratio n/n as follows:

$$n ( {SiCl}_{4} ) : n ( ChCl ) = 1 : 3 - 5$$

The pH value of this solution is obviously very low, below 0.5. Thus obtained solution of ortho-silicic acid (H₄SiO₄) complex with ChCl is further neutralized with:
(i) calcium carbonate (CaCO₃) or betaine [(CH₃)₃N⁺CH₂COO⁻]; and,
(ii) eventually one further “osmolyte” selected from the group comprising: glycine (Gly), taurine, carnitine, inositol, ethanolamine, and their phosphates; mono sugars, such as glucose, xylose, and sorbitol; see patent claims on p. 7 and 8.
   The document shows that SiCl₄ can be hydrolysed with water in a 75% aqueous solution of ChCl yielding a solution that is further neutralized with CaCO₃ or betaine. A serious disadvantage of the compositions according to WO 03/077657 is that it requires the use of very toxic and corrosive silicon tetrachloride (SiCl₄) as a silicon precursor which inevitably releases a significant amount of hydrochloric acid (HCl) that must be neutralised to a certain extent with some suitable base, e.g., with CaCO₃, as shown above.

WO 95/21124 discloses a liquid formulation of stabilized ortho-silicic acid (H₄SiO₄), at a concentration equivalent to 1-8% silicon (Si) content, prepared by hydrolysis of silicon tetrachloride (SiCl₄) as starting silicon compound in a mixture of water, choline chloride (ChCl) and hydrochloric acid (HCl), followed by partial neutralization of thus obtained solution of H₄SiO₄ and HCl with sodium hydroxide (NaOH), followed by addition of glycerol. Said solution has a pH value from 1-4; see patent claims on p. 3. Since the final product is an acidic pH value of 1-3 or “lower than 4”, it is clear to those skilled in the art that this liquid stabilized silicic acid solution contains:
(i) ortho-silicic acid (H₄SiO₄);
(ii) choline chloride (ChCl);
(iii) sodium chloride (NaCl); formed from an excess of HCl and the sodium hydroxide (NaOH) employed as the neutralization agent;
(iv) glycerol; as “a polyalcohol diluent”; and
(v) ad 100% w/w purified water from the starting ChCl-aqueous solution.
   Similar to WO 03/077657, a serious disadvantage of the compositions according to WO 95/21124 is that it involves the use of SiCl₄ which is toxic and highly corrosive substance that requires neutralization with some base, herein NaOH, and thus inevitable generation of the corresponding chloride salt, herein NaCl. The latter additionally destabilizes the silicic acid solution due to the unnecessary increase of ionic strength of the resulting solution.

The objective of the present invention, generally stated, is to provide new and improved ways of stabilizing silicic acid solutions. More in particular, the objective of the invention is to provide stabilized silicic acid solutions that do not suffer from any or all of the drawbacks associated with the prior art composition, such as the use of highly toxic silicon precursors, e.g., SiCl₄ or TEOS, or unnecessary bases that generates useless salts such as NaCl or CaCl₂), which moreover contribute to the instability of resulting silicic acid solution.

### SUMMARY OF THE DISCLOSURE

The present invention, generally stated, resides in the finding that the above-stated objectives can, at least in part, be realized by the stabilising effect of amino acid hydrogensulfates 1, sodium and/or potassium amino acid sulfate 2a,b and sodium and/or potassium hydrogensulfate (MHSO₄, where M=Na and/or K) on the silicic acid solution.

Thus, the present invention, in a first aspect, concerns a stabilized silicic acid solution comprising:
(i) silicic acid, in the form of a mixture of:
   ortho-silicic acid (H₄SiO₄), its
   water-soluble oligomers, and
   water-soluble polymers,
   in a percentage equivalent to 0.01-1.00% w/w silicon (Si),
(ii) one or more amino acid hydrogensulfates 1AA, 1b, and 1c, at a total amount of 0.015-21.53% w/w,

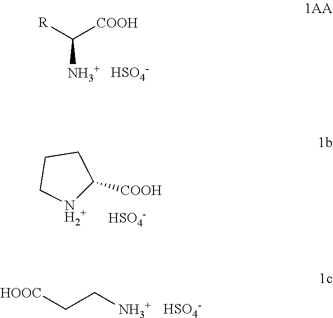

(iii) one or more sodium and/or potassium amino acid sulfates 2aAA, 2bAA, 2c, 2d, 2e, and 2f, at a total amount of 0.00-24.24% w/w,

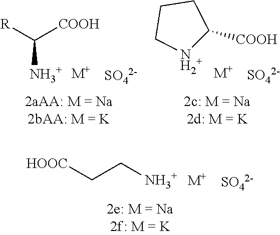

(iv) sodium and/or potassium hydrogensulfate (MHSO₄, where M=Na and/or K), at an amount of 0.00-9.70% w/w,
(v) a diol, selected from the group consisting of 1,2-propylene glycol (3), polyethylene glycol (PEG; 4),

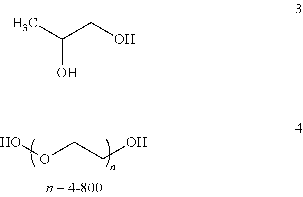

or their mixtures in a relative weight ratio:

$$w ( 3 ) : w ( 4 ) = 1. - 99. : 99. - 1. ,$$

in a percentage of 5-50% w/w within the composition, and
(vi) water, up to 100% w/w of the composition,
   with the proviso that at least one of (iii) and (iv) is present.

In a preferred embodiment, the invention concerns a stabilized silicic acid solution, wherein the one or more amino acids of 1AA, 2aAA and/or 2bAA is/are selected from the group consisting of glycine (Gly), alanine (Ala), valine (Val), norvaline (Nva), leucine (Leu), isoleucine (Ile), norleucine (Nle), serine (Ser), threonine (Thr), aspartic acid (Asp), glutamic acid (Glu), phenylglycine (Phg), phenylalanine (Phe), tyrosine (Tyr), tryptophan (Trp), histidine (His), arginine (Arg), asparagine (Asn), glutamine (Gln), ornithine (Orn), lysine (Lys), methionine (Met), cysteine (Cys), citrulline (Cit) and salts thereof.

In another preferred embodiment, a stabilized silicic acid solution according to the invention, wherein the term ‘water-soluble oligomer’ refers to linear chain, branched-chain, or cyclic oligomers formed by polycondensation of 2-12 ortho-silicic acid (OSA) molecules and the term ‘water-soluble polymer’ refers to polymers of general formula [SiOₓ(OH)₄₋₂ₓ]ₙ, wherein x=1-2, selected from the group consisting of:
sub-nano silicic acid (SNSA) of particles size 0.3-0.6 nm, wherein n=12-20 and M=700-1,400 [g/mol];
sub-nano (or subcolloidal) condensed silicic acid (SNCSA) of particles size 0.6-3 nm, wherein n=20-300 and M=1,400-2,000 [g/mol]; and,
larger SNCSA silicic acid of particles size 3-5 nm, wherein n=300-2,000 and M=20,000-140,000 [g/mol].

Preferably, in the stabilized silicic acid solution, the term ‘water-soluble’ means that a clear water phase can be formed comprising said oligomers and/or polymers at a level of at least 10 ppm, preferably at least 100 ppm, more preferably at least 1000 ppm, at room temperature using plain water at neutral pH.

The above-stated objectives can are realized in a particularly advantageous manner by the stabilising effect of glycine hydrogensulfate (1), sodium and/or potassium glycine sulfate (2a,b), and sodium and/or potassium hydrogensulfate (MHSO₄, where M=Na and/or K) on the silicic acid solution.

Thus, the present invention, in a particularly preferred embodiment, concerns a stabilized silicic acid solution comprising or consisting of:
(i) silicic acid, in the form of a mixture of:
   ortho-silicic acid (H₄SiO₄; OSA), its
   water-soluble oligomers, and
   water-soluble polymers;
(ii) glycine hydrogensulfate (1);

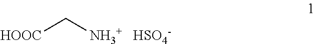

(iii) optionally, sodium and/or potassium glycine sulfate (2a,b);

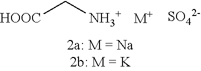

(iv) optionally, sodium and/or potassium hydrogensulfate (MHSO₄, where M=Na and/or K);
(v) a diol, selected from the group consisting of 1,2-propylene glycol (3), polyethylene glycol (PEG; 4);

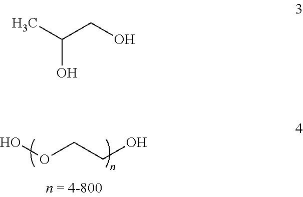

and their mixtures; and
(vi) water,
with the proviso that the composition comprises at least one of (iii) and (iv).

In another particularly preferred embodiment, the present invention concerns a stabilized silicic acid solution comprising or consisting of:
(i) silicic acid, in the form of a mixture of ortho-silicic acid (H₄SiO₄; OSA), its water-soluble oligomers, and water-soluble polymers, at a relative amount of 0.01-1.00 parts (by weight) of silicon (Si),
(ii) glycine hydrogensulfate (1), at a relative amount of 0.015-6.17 parts (by weight),

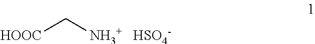

(iii) sodium and/or potassium glycine sulfate (2a,b), at a relative amount of 0.00-5.64 parts (by weight),

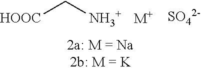

(iv) sodium and/or potassium hydrogensulfate (MHSO₄, where M=Na and/or K), at a relative amount of 0.00-9.70 parts (by weight), and
(v) a diol, selected from the group consisting of 1,2-propylene glycol (3), polyethylene glycol (PEG; 4),

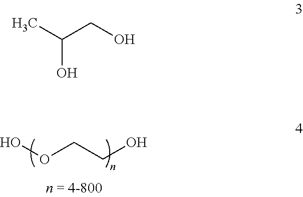

and their mixtures, at a relative amount of 5-50 parts (by weight), in
(vi) water, typically in an amount of water adequate to form a solution,
with the proviso that the composition comprises at least one of (iii) and (iv).

In another preferred embodiment, the present invention concerns a stabilized silicic acid solution comprising or consisting of:
(i) silicic acid, in the form of a mixture of ortho-silicic acid (H₄SiO₄; OSA), its water-soluble oligomers, and water-soluble polymers, in a percentage equivalent to 0.01-1.00% w/w silicon (Si),
(ii) glycine hydrogensulfate (1), 0.015-6.17% w/w,

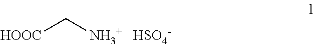

(iii) sodium and/or potassium glycine sulfate (2a,b), 0.00-5.64% w/w,

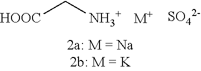

(iv) sodium and/or potassium hydrogensulfate (MHSO₄, where M=Na and/or K), 0.00-9.70% w/w,
(v) a diol, selected from the group consisting of 1,2-propylene glycol (3), polyethylene glycol (PEG; 4),

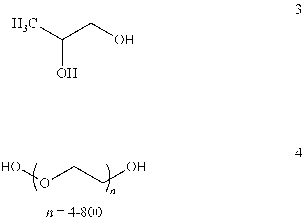

and their mixtures in a relative weight ratio:

$$w ( 3 ) : w ( 4 ) = 1. - 99. : 99. - 1. ,$$

in a percentage of 5-50% w/w within the composition, and
(vi) water, up to 100% w/w of the composition,
with the proviso that:
(a) if (iii) is 0.00% w/w then the percentage of (iv) is >0.00%, and vice versa, and/or
(b) (iii) and (iv) are not both 0.00% w/w.

Preferably, the stabilized silicic acid solution according to the present invention comprises or consists of:
(i) silicic acid, equivalent to 0.10-0.70% w/w silicon (Si),
(ii) glycine hydrogensulfate (1), 0.15-4.32% w/w,
(iii) sodium and/or potassium glycine sulfate (2), 0.00-3.95% w/w,
(iv) sodium and/or potassium hydrogensulfate (MHSO₄, where M=Na and/or K), 0.30-3.62% w/w,
(v) a diol, 5-45% w/w, and
(vi) water, up to 100% w/w of the composition.

More preferably, the stabilized silicic acid solution according to the present invention comprises or consists of:
(i) silicic acid, equivalent to 0.40-0.60% w/w silicon (Si),
(ii) glycine hydrogensulfate (1), 0.62-3.70% w/w,
(iii) sodium and/or potassium glycine sulfate (2), 0.00-3.38% w/w,
(iv) sodium and/or potassium hydrogensulfate (MHSO₄, where M=Na and/or K), 1.05-3.10% w/w,
(v) a diol, 30-45% w/w, and
(vi) purified water, up to 100% w/w of the composition.

The stabilized silicic acid solution according to the present invention is made from the following starting materials:
(I) solid or liquid sodium and/or potassium silicate, xM₂O·ySiO₂, where M=Na and/or K, x=1 and y=1-4,
(II) glycine (5),

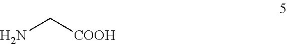

(III) sulfuric acid (H₂SO₄),
(IV) a diol, and
(V) purified water;
where:
(a) a molar ratio, n/n, of silicon (Si) versus glycine (5; Gly) is:

$$n ( Si ) : n ( 5 ) = 1 : 0.8 - 2.4 ,$$

(b) the amount of substance (n) of sulfuric acid equals the sum of the following:
   the amount substance (n) of sodium and/or potassium (Na and/or K) from silicate; and
   25-100% the amount of substance (n) of glycine (5):

$$n ( {H}_{2} {SO}_{4} ) = n ( M ) + 0.25 - 1. \cdot n ( 5 ) ,$$

(c) the product does not contain an excess of sulfuric acid or any other additional acid; and
(d) the pH value of the resulting composition is between 0.35-3.00.

In preferred embodiments, the PEG (4) is selected from the group consisting of: PEG 200, PEG 300, PEG 400, PEG 600, PEG 1000, PEG 1500, PEG 2000, PEG 3000, PEG 3350, PEG 4000, PEG 6000, PEG 8000, PEG 10000, PEG 12000, PEG 20000, PEG 35000 and mixtures thereof.

The invention also concerns a process for the preparation of the composition from the present invention which includes the following manufacturing steps:
A. dissolution of one or more amino acids, preferably glycine, (5) in a mixture of a diol and purified water; which is realized by stirring at a temperature of 10-50° C. during 1-30 minutes,
B. separate stepwise addition of sulfuric acid (H₂SO₄) to one part of purified water at a temperature of 5-20° C. during 5-30 minutes,
C. addition of the sulfuric acid solution from step B into the solution from step A, at a temperature of 5-20° C. during 1-30 minutes,
D. separate preparation of sodium and/or potassium silicate solution in water either by:
   the dissolution of solid substances in a part of purified water, or
   dilution of liquid substances with a part of purified water, and
E. a dropwise addition of the solution from step D to a cooled solution from step C, at a temperature of 5-15° C. with an intensive stirring during 5-60 minutes,
yielding the final liquid silicic acid composition according to the present invention.

Preferably, the one or more amino acids are selected from the group consisting of glycine (Gly), alanine (Ala), valine (Val), norvaline (Nva), leucine (Leu), isoleucine (Ile), norleucine (Nle), serine (Ser), threonine (Thr), aspartic acid (Asp), glutamic acid (Glu), phenylglycine (Phg), phenylalanine (Phe), tyrosine (Tyr), tryptophan (Trp), histidine (His), arginine (Arg), asparagine (Asn), glutamine (Gln), ornithine (Orn), lysine (Lys), methionine (Met), cysteine (Cys), citrulline (Cit), proline (Pro), beta-alanine (βAl) and salts thereof.

More preferably, in the process of the invention:
(a) the molar ratio, n/n, of silicon (Si) to amino acid (AA) is:

$$n ( Si ) : n ( AA ) = 1 : 0.8 - 2.4 ,$$

(b) the amount of substance (n) of sulfuric acid equals the sum of the following amounts:
   the amount of substance (n) of sodium and/or potassium (Na and/or K) from silicate; and
   25-100% of the amount of substance (n) of the corresponding amino acid (AA):

$$n ( {H}_{2} {SO}_{4} ) = n ( M ) + 0.25 - 1. \cdot n ( AA ) ,$$

and/or,
(c) sodium and/or potassium silicate, xM₂O·ySiO₂, where M=Na and/or K, used in the process is:
   in the solid form with 97-99% w/w sodium or potassium silicate, or
   in the form of a liquid of a concentration of about 24-47% w/w sodium or potassium silicate,
   which is characterized by the following molar ratio, x:y, n/n: x=1.00, y=1.00; x=1.00, y=1.60-1.70; x=1.00, y=2.00-2.10; x=1.00, y=3.30-3.40; x=1.00, y=3.90-4.00; or mixtures of these substances.

Preferably, in the process of the invention, the one or more amino acids are or include histidine (His), arginine (Arg), ornithine (Orn), and/or lysine (Lys), wherein the side-moiety (R) is in the form of the free base, and the amount of substance (n) of sulfuric acid used in the process is increased for an equimolar amount of said employed AA, to ensure the conversion of the respective side-chain basic functional group into the corresponding hydrogensulfate salt.

The composition according to the present invention is used as a food supplement, functional food ingredient, feed additive, functional feed additive, biostimulant for aquaculture, active cosmetic ingredient (ACI), or as an active pharmaceutical ingredient (API) that provides highly bioavailable silicon (Si) and its beneficial nutritional and pharmacological effects.

Alternatively, the composition from the present invention is used as an agrochemical product for silicon fertilization, plant growth-boosting, and plant protection.

### DESCRIPTION OF FIGURES

FIG. 1 depicts the polymerisation of ortho-silicic acid (OSA) to various said silicic acid oligomers, polymers, and finally silica gel. S2=dimetric silicic acid; S3=trimeric silicic acid; bS4=branched tetrameric silicic acid; cS3=cyclic tetrameric silicic acid; SS=cage-type sesquisiloxane type oligomeric silicic acid; SNSA=sub-nano silicic acid; SNCSA=sub-nano condensed silicic acid; PSNP=polymerised silica nano-particles; SG=silica gel.

FIG. 2 depicts possible structure of the molecular complex of ortho-silicic acid (OSA) with glycine hydrogensulfate (1), potassium glycine sulfate (2b), sodium and/or potassium hydrogensulfate (MHSO₄, M=Na and/or K), 1,2-propylene glycol (3) and water (H₂O) at stoichiometric ratio OSA:glycine=1:1, stabilized by multiple hydrogen bonds.

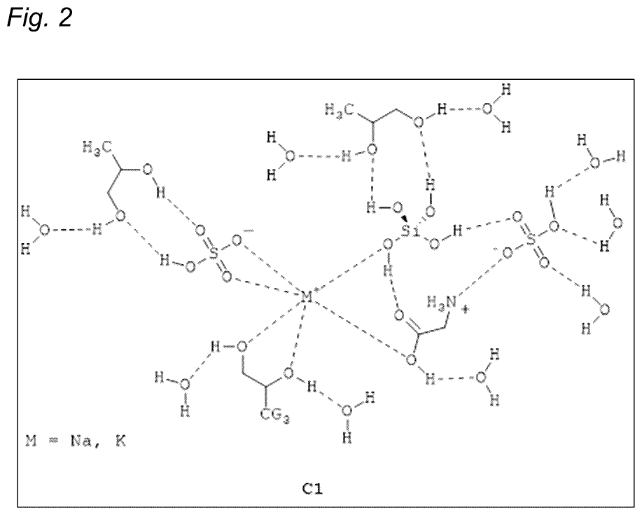

### DETAILED DESCRIPTION OF THE INVENTION

The present invention, in a first aspect, concerns a stabilized silicic acid solution comprising:
(i) silicic acid, in the form of a mixture of:
   ortho-silicic acid (H₄SiO₄; OSA), its
   water-soluble oligomers, and
   water-soluble polymers
(ii) one or more amino acid hydrogensulfates 1AA, 1b, 1c,

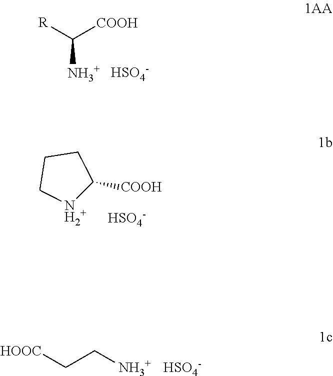

(iii) optionally one or more sodium and/or potassium amino acid sulfates 2aAA, 2bAA, 2c, 2d, 2e, 2f,

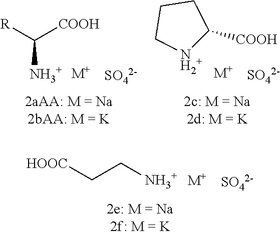

(iv) optionally sodium and/or potassium hydrogensulfate (MHSO₄, where M=Na and/or K),
(v) a diol, selected from the group consisting of 1,2-propylene glycol (3), polyethylene glycol (PEG; 4),

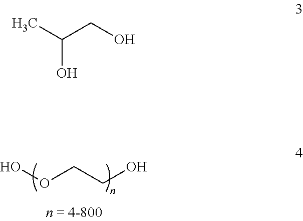

or their mixtures in a relative weight ratio:

$$w ( 3 ) : w ( 4 ) = 1. - 99. : 99. - 1. ,$$

and
(vi) water,
with the proviso that at least one of (iii) and (iv) is present.

In a preferred embodiment of the invention, a stabilized silicic acid solution comprising:
(i) silicic acid, in the form of a mixture of:
   ortho-silicic acid (H₄SiO₄), its
   water-soluble oligomers, and
   water-soluble polymers,
   in a percentage equivalent to 0.01-1.00% w/w silicon (Si),
(ii) one or more amino acid hydrogensulfates 1AA, 1b, 1c, at a total amount of 0.015-21.53% w/w,

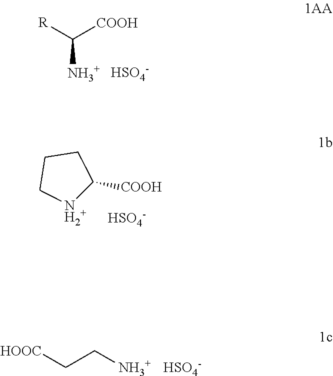

(iii) one or more sodium and/or potassium amino acid sulfates 2aAA, 2bAA, 2c, 2d, 2e, 2f, at a total amount of 0.00-24.24% w/w,

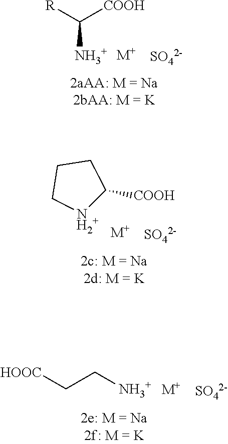

(iv) sodium and/or potassium hydrogensulfate (MHSO₄, where M=Na and/or K), at an amount of 0.00-9.70% w/w,
(v) a diol, selected from the group consisting of 1,2-propylene glycol (3), polyethylene glycol (PEG; 4),

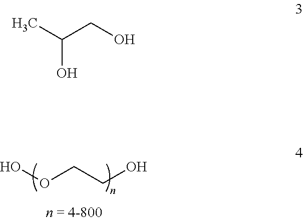

or their mixtures in a relative weight ratio:

$$w ( 3 ) : w ( 4 ) = 1. - 99. : 99. - 1. ,$$

in a percentage of 5-50% w/w within the composition, and
(vi) water, up to 100% w/w of the composition,
with the proviso that at least one of (iii) and (iv) is present.

In a preferred embodiment, a stabilized silicic acid solution according to the invention is provided, wherein the amino acids of 1AA, 2aAA and/or 2bAA are selected from the group consisting of glycine (Gly), alanine (Ala), valine (Val), norvaline (Nva), leucine (Leu), isoleucine (Ile), norleucine (Nle), serine (Ser), threonine (Thr), aspartic acid (Asp), glutamic acid (Glu), phenylglycine (Phg), phenylalanine (Phe), tyrosine (Tyr), tryptophan (Trp), histidine (His), arginine (Arg), asparagine (Asn), glutamine (Gln), ornithine (Orn), lysine (Lys), methionine (Met), cysteine (Cys), citrulline (Cit) and salts thereof.

In a particularly preferred embodiment of the invention, a stabilized silicic acid solution is provided comprising or consisting of:
(i) silicic acid, in the form of a mixture of:
   ortho-silicic acid (H₄SiO₄; OSA), its
   water-soluble oligomers, and
   water-soluble polymers;
(ii) glycine hydrogensulfate (1);

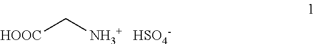

(iii) optionally sodium and/or potassium glycine sulfate (2a,b);

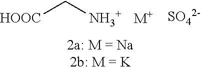

(iv) optionally sodium and/or potassium hydrogensulfate (MHSO₄, where M=Na and/or K); and
(v) a diol, selected from the group consisting of 1,2-propylene glycol (3) and/or polyethylene glycol (PEG; 4);

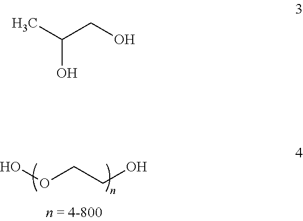

and their mixtures; and
(vi) purified water;
with the proviso that the composition comprises at least one of (iii) and (iv).

In another particularly preferred embodiment, the present invention concerns a stabilized silicic acid solution comprising or consisting of:
(i) silicic acid, in the form of a mixture of ortho-silicic acid (H₄SiO₄; OSA), its water-soluble oligomers, and water-soluble polymers, at a relative amount of 0.01-1.00 parts (by weight) of silicon (Si),
(ii) glycine hydrogensulfate (1), at a relative amount of 0.015-6.17 parts (by weight),

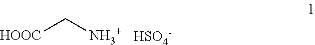

(iii) sodium and/or potassium glycine sulfate (2a,b), at a relative amount of 0.00-5.64 parts (by weight),

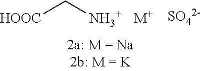

(iv) sodium and/or potassium hydrogensulfate (MHSO₄, where M=Na and/or K), at a relative amount of 0.00-9.70 parts (by weight), and
(v) a diol, selected from the group consisting of 1,2-propylene glycol (3), polyethylene glycol (PEG; 4),

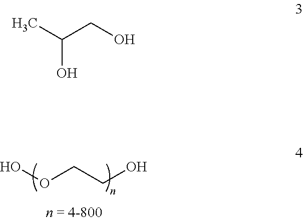

and their mixtures, at a relative amount of 5-50 parts (by weight), in
(vi) water, typically in an amount of water adequate to form a solution,
with the proviso that the composition comprises at least one of (iii) and (iv).

Amounts expressed as ‘% w/w’ refer to the weight of a given component relative to the total weight of the stabilized silicic acid-containing solution, unless specifically indicated otherwise.

As used herein the term ‘water-soluble’ when relating to silicic acid compounds in the context of this application refers to the property that said compound can be dissolved in water, even if, in given instances, it may be sparingly and rather slowly. Typically, it means that a clear water phase can be formed comprising said compound at a level of at least 10 ppm, preferably at least 100 ppm, more preferably at least 1000 ppm, at room temperature. It should be noted, that solubility is pH dependent, and, in accordance with the present definitions, solubility is given for, and is determined using, plain water, i.e., water at neutral pH, such as a pH of about 7.

In accordance with the present invention, the compositions preferably comprise silicic acid oligomers and polymers in sub-colloidal form. Solutions comprising such subcolloidal particles typically pass through a 0.1-micron filter. In particularly preferred embodiments of the invention, the water-soluble silicic acid oligomers and polymers are in the form of sub-colloidal particles having a size within the range of 0.1-10 nm, more preferably within the range of 0.2-8 nm, still more preferably 0.3-6 nm, most preferably 0.3-5 nm.

Particle size determinations can be made using ²⁹Si NMR spectroscopy, TEM, and/or SEM. In preferred embodiments of the invention, at least 50% of the silicic acid-containing particles in the compositions have a particle diameter within the aforementioned size ranges, more preferably at least 60%, still more preferably at least 70%, still more preferably at least 75%, still more preferably at least 80%, still more preferably at least 85%, still more preferably at least 90%, still more preferably at least 95%, still more preferably at least 97%, still more preferably at least 98%, most preferably at least 99%.

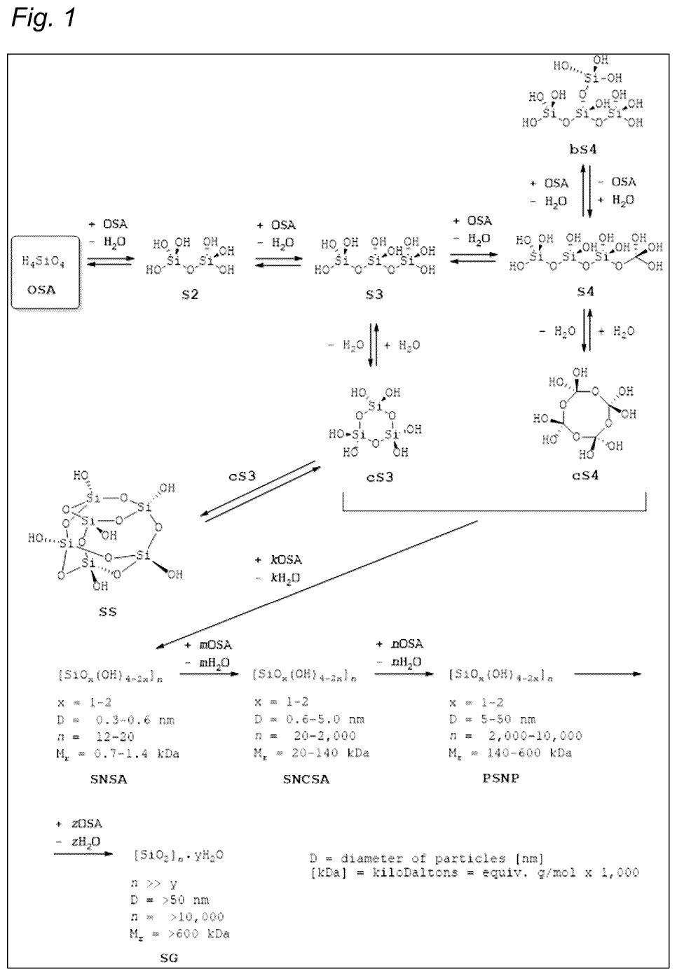

In particularly preferred embodiments of the invention, the term ‘water-soluble oligomer’ refers to linear chain, branched-chain, or cyclic oligomers formed by polycondensation of 2-12 ortho-silicic acid (OSA) molecules such as S2, S3, bS4, cS4, SS, and similar oligomers according to FIG. 1.

As used herein the term ‘water-soluble polymer’ means polymers of general formula [SiOₓ(OH)₄₋₂ₓ]ₙ, X=1-2, such as:
sub-nano silicic acid (SNSA) of particles size 0.3-0.6 nm, n=12-20 and M=700-1,400 [g/mol]; which is freely water-soluble;
sub-nano (or subcolloidal) condensed silicic acid (SNCSA) of particles size 0.6-3 nm, n=20-300 and M=1,400-2,000 [g/mol], which is freely water-soluble; and,
larger SNCSA silicic acid of particles size 3-5 nm, n=300-2,000 and M=20,000-140,000 [g/mol]; which is still freely or moderately water-soluble, but may eventually form slight opalescence of the solution upon prolonged storage.

In preferred embodiments of the invention, the silicic acid oligomers and polymers are water-soluble, and, at total concentrations of silicon within the range of 0.01-1.00% w/w, essentially clear solutions can be produced. Solutions that, after long-term storage, turn slightly opalescent are encompassed by the invention. These solutions are stable and not prone to significant further polymerisation which would be accompanied by gelling, and the formation of a gel.

In preferred embodiments, (i) is present in a percentage equivalent to 0.01-1.00% w/w silicon (Si), more preferably 0.10-0.70% w/w, most preferably 0.40-0.60% w/w.

In preferred embodiments, (ii) is present in an amount of 0.015-6.17% w/w, based on the total weight of the silicic acid-containing solution, more preferably 0.15-4.32% w/w, most preferably 0.62-3.70% w/w.

In preferred embodiments, (iii) is present in an amount of 0.00-5.64% w/w, based on the total weight of the silicic acid-containing solution, more preferably 0.00-3.95% w/w, most preferably 0.00-3.38% w/w.

The relative amounts of (iii) and (iv) typically are inversely proportional and their absolute values depend on the amount of sulfuric acid employed for the neutralization of a mixture of amino acid, preferably glycine, and sodium and/or potassium silicate in the process disclosed below. The higher relative amount of (iii) means the lower relative amount of (iv) and vice versa. In the extreme case if (iii) is 0.00% w/w, then the percentage of (iv) is of the largest relative value, in any case>0.00% w/w, within the defined stoichiometry of sulfuric acid employed for the process described below. In another extreme case, if (iv) is 0.00% w/w, then (iii) is the largest relative value, whose absolute value depends on the stoichiometric ratio of sulfuric acid employed in the process disclosed below. In this manner, there is no case where both (iii) and (iv) are 0.00% w/w.

In preferred embodiments, (iv) is present in an amount of 0.00-9.70% w/w, based on the total weight of the silicic acid-containing solution, more preferably 0.30-3.62% w/w, most preferably 1.05-3.10% w/w.

In preferred embodiments, (v) is present in an amount of 5-50% w/w, based on the total weight of the silicic acid-containing solution, more preferably 10-45% w/w, most preferably 30-45% w/w.

In preferred embodiments, 1,2-propylene glycol (3) is used as the only diol. In the case where a mixture of 3 and 4 are employed, then a preferable weight ratio of these ingredients is from 1/99% w/w to 99/1% w/w, more preferably from 10/90% w/w to 90/10% w/w, most preferably from 20/80% w/w to 80/20% w/w.

In preferred embodiments of the invention, the water is selected from the group consisting of tap water, demineralized water, deionized water, distilled water, reverse osmosis purified water, or water that has been purified in any other way, such as to render it substantially without free ions. In preferred embodiments of the invention, the water is demineralized water, deionized water, distilled water, or purified water. In one particularly preferred embodiment of the invention, the water is purified water according to Ph.Eur. or similar pharmacopoeial standards.

Preferably, the composition of a stabilized silicic acid solution according to the present invention comprises or consists of:
(i) silicic acid in an amount equivalent to 0.01-1.00% w/w silicon (Si);
(ii) amino acid hydrogensulfate, preferably glycine hydrogensulfate (1), in an amount within the range of 0.015-6.17% w/w;
(iii) sodium and/or potassium amino acid sulfate, preferably sodium and/or potassium glycine sulfate (2), in an amount within the range of 0.00-5.64% w/w;
(iv) sodium and/or potassium hydrogensulfate (MHSO₄, where M=Na or K), in an amount within the range of 0.00-9.70% w/w;
(v) a diol in an amount within the range of 5-50% w/w; and
(vi) water, up to 100% w/w of the composition;
providing that:
(a) if (iii) is 0.00% w/w then the percentage of (iv) is >0.00%, and vice versa, and/or
(b) (iii) and (iv) are not both 0.00% w/w.

More preferably, the composition of a stabilized silicic acid solution according to the present invention comprises or consists of:
(i) silicic acid, in an amount equivalent to 0.10-0.70% w/w silicon (Si);
(ii) amino acid hydrogensulfate 1AA, 1b, 1c, preferably glycine hydrogensulfate (1), in an amount within the range of 0.15-4.32% w/w;
(iii) sodium and/or potassium amino acid sulfate 2aAA, 2bAA, 2c, 2d, 2e, 2f, preferably sodium and/or potassium glycine sulfate (2), in an amount within the range of 0.00-3.95% w/w;
(iv) sodium and/or potassium hydrogensulfate (MHSO₄, where M=Na and/or K) in an amount within the range of 0.30-3.62% w/w;
(v) a diol in an amount within the range of 10-45% w/w; and
(vi) water, up to 100% w/w of the composition.

Most preferably, the composition of a stabilized silicic acid solution according to the present invention comprises or consists of:
(i) silicic acid in an amount equivalent to 0.40-0.60% w/w silicon (Si);
(ii) amino acid hydrogensulfate 1AA, 1b, 1c, preferably glycine hydrogensulfate (1), within the range of 0.62-3.70% w/w;
(iii) sodium and/or potassium amino acid sulfate 2aAA, 2bAA, 2c, 2d, 2e, 2f, preferably sodium and/or potassium glycine sulfate (2), within the range of 0.00-3.38% w/w;
(iv) sodium and/or potassium hydrogensulfate (MHSO₄, where M=Na and/or K) within the range of 1.05-3.10% w/w;
(v) a diol within the range of 30-45% w/w; and
(vi) water, up to 100% w/w of the composition.

In a further preferred embodiment, the present invention concerns a stabilized silicic acid solution comprising or consists of:
(i) silicic acid, in the form of a mixture of ortho-silicic acid (H₄SiO₄; OSA), its water-soluble oligomers, and water-soluble polymers, at a relative amount of 0.01-1.00 parts (by weight) of silicon (Si), more preferably 0.10-0.70 parts (by weight), most preferably 0.40-0.60 parts (by weight),
(ii) amino acid hydrogensulfate 1AA, 1b, 1c, preferably glycine hydrogensulfate (1), at a relative amount of 0.015-6.17 parts (by weight), more preferably 0.15-4.32 parts (by weight), most preferably 0.62-3.70 (parts by weight),

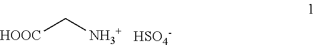

(iii) sodium and/or potassium amino acid sulfate 2aAA, 2bAA, 2c, 2d, 2e, 2f, preferably sodium and/or potassium glycine sulfate (2a,b), at a relative amount of 0.00-5.64 parts (by weight), more preferably 0.00-3.95 parts (by weight), most preferably 0.00-3.38 parts (by weight),

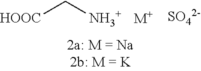

(iv) sodium and/or potassium hydrogensulfate (MHSO₄, where M=Na and/or K), at a relative amount of 0.00-9.70 parts (by weight), more preferably 0.30-3.62 parts (by weight), most preferably 1.05-3.10 parts (by weight), and
(v) a diol, selected from the group consisting of 1,2-propylene glycol (3), polyethylene glycol (PEG; 4),

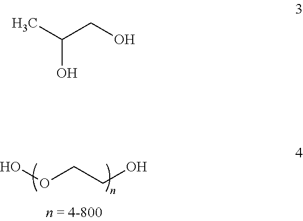

and their mixtures, at a relative amount of 5-50 parts (by weight), more preferably 10-45 parts (by weight), most preferably 30-45 parts (by weight),
(vi) in water, typically in a quantity of water sufficient to form a solution.

The stabilized silicic acid solution according to the present invention is typically made from the following starting materials:
(i) solid or liquid sodium and/or potassium silicate, xM₂O·ySiO₂, where M=Na and/or K, x=1 and y=1-4,
(ii) amino acid, preferably glycine (5),

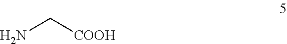

(iii) sulfuric acid (H₂SO₄),
(iv) a diol, preferably a diol as define herein before, and
(v) water;
wherein:
(a) a molar ratio, n/n, of silicon (Si) versus amino acid, preferably glycine (5), is:

$$n ( Si ) : n ( 5 ) = 1 : 0.8 - 2.4 ,$$

(b) the amount of substance (n) of sulfuric acid equals to the sum of the following:
   the amount substance (n) of sodium and/or potassium (Na and/or K) from silicate; and
   25-100% the amount of substance (n) of amino acid, preferably glycine (5):

$$n ( {H}_{2} {SO}_{4} ) = n ( M ) + 0.25 - 1. \cdot n ( 5 ) ,$$

(c) the product does not contain an excess of sulfuric acid or any other additional acid; and
(d) the pH value of the resulting composition is between 0.35-3.00.

In accordance with preferred embodiments, sodium and/or potassium silicate, xM₂O·ySiO₂, where M=Na and/or K, that is employed for the preparation of the composition from the present invention is:
in the solid form with 97-99% w/w sodium or potassium silicate, or
in the form of a liquid of a concentration of about 24-47% w/w sodium or potassium silicate,
   which is characterized by the following molar ratio x:y, n/n:

$$x = 1. , y = 1. ;$$

$$x = 1. , y = 1.6 - 1.7 ;$$

$$x = 1. , y = 2. - 2.1 ;$$

$$x = 1. , y = 3.3 - 3.4 ;$$

$$x = 1. , y = 3.9 - 4. ;$$

or mixtures of these substances. Such types of sodium and/or potassium silicates are usually commercially available products worldwide.

In accordance with preferred embodiments, 1,2-propylene glycol (3) that is used for the preparation of the composition from the present invention is a food or pharma-grade purity product, also commercially available worldwide.

In accordance with preferred embodiments, the types of PEGs (4) are selected from the group consisting of: PEG 200, PEG 300, PEG 400, PEG 600, PEG 1000, PEG 1500, PEG 2000, PEG 3000, PEG 3350, PEG 4000, PEG 6000, PEG 8000, PEG 10000, PEG 12000, PEG 20000, PEG 35000, or mixtures of these substances. In accordance with preferred embodiments, the PEG is a food or pharma-grade purity product, which is commercially available worldwide from various manufacturers and distributors.

Sulfuric acid (H₂SO₄) that is used for the preparation of the composition from the present invention can be of various concentrations, most practically from 50% to 96% w/w, preferably of a food or pharma grade quality.

The amino acid(s), preferably glycine (5), that is/are used for the preparation of the composition from the present invention can be of various origins, manufactured synthetically or isolated from animal or plant protein hydrolysates, but is preferably of a food or pharma grade quality. Such grades are widely available from various manufacturers and distributors worldwide.

A second aspect of the present invention concerns a process for preparing a stabilized silicic acid solution, preferably a stabilized silicic acid solution as defined herein before, said process comprises the following steps:
A. dissolution of one or more amino acids in a mixture of a diol and purified water; which is realized by stirring at a temperature of 10-50° C. during 1-30 minutes,
B. separate stepwise addition of sulfuric acid (H₂SO₄) to one part of purified water at a temperature of 5-20° C. during 5-30 minutes,
C. addition of the sulfuric acid solution from step B into the solution from step A, at a temperature of 5-20° C. during 1-30 minutes,
D. separate preparation of sodium or potassium silicate solution in water either by:
   the dissolution of solid substances in a part of purified water, or
   dilution of liquid substances with a part of purified water, and
E. a dropwise addition of the solution from step D to a cooled solution from step C, at a temperature of 5-15° C. with an intensive stirring during 5-60 minutes, yielding the final liquid silicic acid composition.

In a preferred embodiment, a process as defined herein is provided wherein:
(a) the molar ratio, n/n, of silicon (Si) to amino acid (AA) is:

$$n ( Si ) : n ( AA ) = 1 : 0.8 - 2.4 ,$$

(b) the amount of substance (n) of sulfuric acid equals the sum of the following:
   the amount of substance (n) of sodium and/or potassium (Na and/or K) from silicate; and
   25-100% of the amount of substance (n) of the corresponding amino acid (AA):

$$n ( {H}_{2} {SO}_{4} ) = n ( M ) + 0.25 - 1. \cdot n ( AA ) ,$$

and/or
(c) sodium and/or potassium silicate, xM₂O·ySiO₂, where M=Na and/or K, used in the process is:
   in the solid form with 97-99% w/w sodium or potassium silicate, or
   in the form of a liquid of a concentration of about 24-47% w/w sodium or potassium silicate,
   which is characterized by the following molar ratio, x:y, n/n:x=1.00, y=1.00; x=1.00, y=1.60-1.70; x=1.00, y=2.00-2.10; x=1.00, y=3.30-3.40; x=1.00, y=3.90-4.00; or mixtures of these substances.

A process according to claim 12, wherein the one or more amino acids are selected from the group consisting of glycine (Gly), alanine (Ala), valine (Val), norvaline (Nva), leucine (Leu), isoleucine (Ile), norleucine (Nle), serine (Ser), threonine (Thr), aspartic acid (Asp), glutamic acid (Glu), phenylglycine (Phg), phenylalanine (Phe), tyrosine (Tyr), tryptophan (Trp), histidine (His), arginine (Arg), asparagine (Asn), glutamine (Gln), ornithine (Orn), lysine (Lys), methionine (Met), cysteine (Cys), citrulline (Cit), proline (Pro), beta-alanine (βAl) and salts thereof.

In a preferred embodiment, a process as defined herein is provided wherein the one or more amino acids are or include histidine (His), arginine (Arg), ornithine (Orn), and/or lysine (Lys), wherein the side-moiety (R) is in the form of the free base, and the amount of substance (n) of sulfuric acid used in the process is increased for an equimolar amount of said employed AA, to ensure the conversion of the respective side-chain basic functional group into the corresponding hydrogensulfate salt.

In a preferred embodiment, a process for preparing a stabilized silicic acid solution as defined herein is provided, said process comprising the following steps:
A. dissolution of glycine (5) in a mixture of a diol and purified water; which is realized by stirring at a temperature of 10-50° C. during 1-30 minutes,
B. separate stepwise addition of sulfuric acid (H₂SO₄) to one part of purified water at a temperature of 5-20° C. during 5-30 minutes,
C. addition of the sulfuric acid solution from step B into the solution from step A, at a temperature of 5-20° C. during 1-30 minutes,
D. separate preparation of sodium and/or potassium silicate solution in water either by:
   the dissolution of solid substances in a part of purified water, or
   dilution of liquid substances with a part of purified water, and
E. a dropwise addition of the solution from step D to a cooled solution from step C, at a temperature of 5-15° C. with an intensive stirring during 5-60 minutes, yielding the final liquid silicic acid composition according to the present invention.

The reactions that take place during the manufacturing process, using glycine (Gly; 5) as an example, are as follows:
(1) during step C, a neutralisation of glycine with sulfuric acid occurs, yielding glycine hydrogensulfate (1) with an excess of remaining unreacted H₂SO₄; see Equation 1a:
(2) during step E, upon addition of sodium or potassium silicate solution into said glycine hydrogensulfate (1)-sulfuric acid system, a neutralization of sodium or potassium “oxide” from the corresponding silicate take place giving the corresponding sodium or potassium hydrogensulfate and ortho-silicic acid (H₄SiO₄); see Equation 2a:

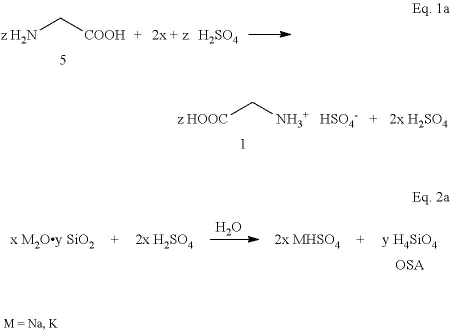

(3) in the case of the use of lower molar excess of sulfuric acid, e.g., if the amount of substance (n) of H₂SO₄ is equal to the amount of substance of sodium (Na) and/or potassium (K) from silicate plus 0.25 less than 1.00, e.g, 0.50 (50%), of the amount of substance of glycine (5), then, there is not enough sulfuric acid for quantitative conversion of glycine base to glycine hydrogensulfate (1). In this case, sodium and/or potassium hydrogensulfate (MHSO₄, M=Na and/or K) partially reacts with an excess of unreacted glycine (free base) yielding a mixed salt sodium or potassium glycine sulfate (2a,b); see equations 1b and 2b:

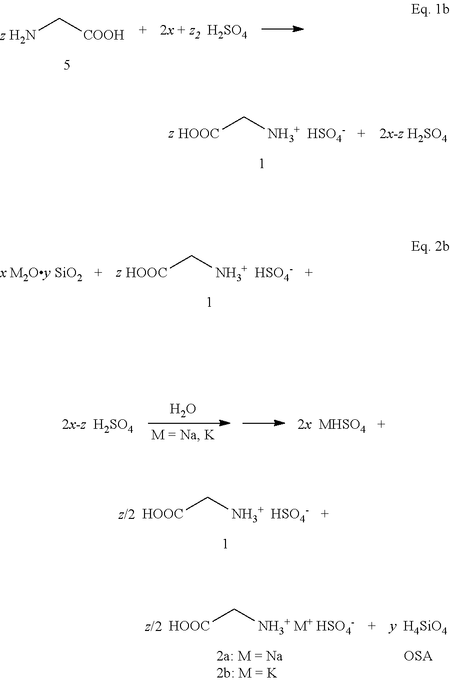

Additionally, if the amount of substance of H₂SO₄ equals to the sum of the amount of substance of sodium (or potassium) from silicate and the amount of substance of glycine, then a mixture of sodium (or potassium) hydrogensulfate and glycine hydrogensulfate (1) is formed. In this case, no mixed salt sodium glycine sulfate (2a) or potassium glycine sulfate (2b) is formed.

The quantitative composition of stabilising ingredients glycine hydrogensulfate (1), sodium or potassium glycine sulfate (2a or 2b) and sodium and/or potassium hydrogensulfate (MHSO₄; M=Na and/or K) can be readily calculated from the amount of sodium or potassium silicate, glycine (5), and sulfuric acid (H₂SO₄) employed, having in mind that sodium or potassium hydroxide (from sodium or potassium silicate) are stronger bases than glycine. In this manner, the first quantity of sulfuric acid is spent on the neutralisation of hydroxide (OH⁻) anions from sodium or potassium silicate to form water (H₂O) and sodium and/or potassium hydrogensulfate (MHSO₄; M=Na and/or K), while all excess of used sulfuric acid is employed for the neutralisation of glycine to its salt, glycine hydrogensulfate (1). In the case if sulfuric acid is used in lower molar excess that is necessary for the neutralisation of both NaOH or KOH (from silicates) and glycine base, then primarily formed sodium and/or potassium hydrogensulfate (MHSO₄; M=Na and/or K) neutralizes an excess of glycine base yielding sodium or potassium glycine sulfate (2a or 2b).

In a preferred embodiment of the invention, the mixture of diol and purified water in step A. comprises the diol and purified water in a ratio of 1:1 to 3:1 w/w.

In a preferred embodiment of the invention, in step A., the amino acid, preferably glycine, (5) is dissolved in said mixture of diol and purified water to a concentration within the range of 0.01-5.00% w/w.

In a preferred embodiment of the invention, in step B., the sulfuric acid of starting concentration of 50-96% w/w is added to part of the purified water to a concentration within the range of 0.01-40.00% w/w.

In a preferred embodiment of the invention, in step D., a sodium or potassium silicate solution is prepared, comprising sodium or potassium silicate at a concentration within the range of 0.02-16.00% w/w.

Typical experimental procedures for the preparation of the products based on the present composition are described in Examples 7-35.

The results of the stability studies of the typical formulations from the present invention in comparison to the formulations that can be considered as the closest prior art solutions (products from comparative Examples 1-6) are described in Example 36.

A third aspect of the present invention concerns a stabilized silicic acid solution that is obtainable in any of the processes as defined herein before. It is the understanding of the present inventors that the compositions obtained by said processes have the characteristics of the products defined herein before, but the invention is not limited by any such theory.

The composition according to the present invention may be used as a food supplement, functional food ingredient, feed additive, functional feed additive, active cosmetic ingredient (ACI), or as an active pharmaceutical ingredient (API) that provides highly bioavailable silicon (Si) and its beneficial nutritional and pharmacological effects.

When used as a food supplement, functional food ingredient, feed additive, functional feed additive, active cosmetic ingredient (ACI), or as active pharmaceutical ingredient (API), the composition from the present invention is used as the functional ingredient which provides beneficial nutritional and pharmacological effects selected from the group consisting of: antioxidant, anti-inflammatory, anti-osteoporotic, stimulation of collagen biosynthesis, stimulation of cartilage and connective tissue formation, stimulation of hair, nails, and skin strength and growth, vitamin D-mimetic activity as it stimulates calcium uptake, stimulation of bone mineralization and growth, stimulation of skin repair and rejuvenation, anti-wrinkle effect, wound-healing effect, immune-system modulating effects, neuroprotective effect by preventing accumulation of aluminium in the brain, anti-atherosclerotic effect, antihypertensive effect, antidiabetic effect, antihistaminic effect, analgesic effect, and diuretic effect.

More precisely, the composition according to the present invention can be used as the active pharmaceutical ingredient for prevention and/or treatment of medical disorders or diseases selected from the group comprising: skin ulcers including decubitus ulcer, a diabetic skin ulcer, a burn ulcer, a traumatic ulcer or a crural ulcer, a surgical site wound or a diabetic gangrene; cancer diseases including melanoma; allergies; osteoporosis, bone fractures, osteoarthritis, and other disorders and diseases of bone and cartilage; prevention and treatment of inflammatory diseases: dermatitis, arthritis, osteoarthritis, otitis including middle ear inflammation, gastritis, gastroenteritis, inflammatory bowel disease including Chron's disease, sinusitis, rhinosinusitis, pharyngitis, laryngitis, lower respiratory tract inflammations and other inflammatory diseases; periodontitis; poor peripheral blood circulation; paresthesia; hypertension; diabetes; atherosclerosis; vertigo; headache; tinnitus; insomnia; diarrhea; constipation; hemorrhoids; kidney stones; urinary infections; lower back pain; excessive female discharge; abscesses and ulcers in the genital area and cervix as well as mastitis; alopecia.

When used as a food supplement, functional food ingredient, functional feed additive, active cosmetic ingredient (ACI), or active pharmaceutical ingredient (API) the composition from the present invention can be used in a dosage equivalent to 0.1-30 mg silicon (Si) per day, preferably in a dosage equivalent to 1-15 mg Si per day, most preferably in a dosage equivalent to 3-12 mg Si per day.

When used as a feed additive, the composition from the present invention is preferably used in dosages equivalent to 0.001-0.5 mg silicon (Si)/kg of the body weight of an animal per day, preferably 0.01-0.2 mg Si/kg/day, most preferably 0.05-0.15 mg Si/kg/day.

Alternatively, the composition from the present invention can be used as an agrochemical product for silicon fertilization, plant growth-boosting, and plant protection.

More precisely, the composition from the present invention can be used as the active agrochemical agent that boosts plant growth and increases crop yield, improves strength, decreases climate stress including resistance to drought, provides resistance to mineral stress, and increases resistance to attacks by insect pests, nematodes, and diseases including fungal diseases.

When used as an agrochemical product, the composition from the present invention is preferably used in a concentration providing 5-500 ppm of silicon (Si), depending on the application type, which may e.g. involve fertigation or foliar spray, such as 10-100 ppm or 25-50 ppm.

Alternatively, the composition from the present invention can be used in aquaculture, typically as a fertilizer, as a feed additive and/or as a biostimulant, so as to improve productivity and/or reduce the environmental impact thereof.

More precisely, the composition from the present invention can be used in aquaculture with one or more of the following objectives and/or results:
enhancing growth of the aquatic animal;
accelerating growth of the aquatic animal;
increasing the weight of the aquatic animal;
reducing the time to harvest;
improving the overall yield;
improving feed utilization;
increasing the feed conversion rate;
improving the water quality;
increasing dissolved oxygen level and/or maintaining dissolved oxygen levels;
decreasing the ammonia concentration and/or preventing rises in ammonia concentration;
improving the pH of the water and/or maintaining the pH at appropriate levels;
decreasing salinity and/or preventing salinization and/or hypersalinization;
enhancing/stimulating growth of phytoplankton, especially the diatoms in the water; and
reducing the ecological impact of aquaculture.

When used in aquaculture, the composition of the present invention is preferably added to the water in which the aquatic species is kept, at dosages resulting in a level of at least 0.1 ppm of silicon (Si) in the water, preferably at least 0.5 ppm, at least 1 ppm, at least 2.5 ppm, at least 5 ppm, at least 10 ppm, or at least 25 ppm. Alternatively, the composition of the present invention may be added to the feed, typically in quantities resulting in a silicon (Si) level in the feed of at least 0.001 ppm, preferably at least 0.005 ppm, at least 0.01 ppm, at least 0.025 ppm, at least 0.05 ppm, at least 0.10 ppm, or at least 0.25 ppm.

As will be evident to the skilled person, based on the present teachings, many of the envisaged uses, such as those defined here above, will entail the dilution of the stabilized silicic acid solution of the present invention, typically by the mere addition of water, before e.g., application to a crop or before administration to an animal. Hence, a further aspect of the invention concerns a method of providing a dilute and/or ready-to-use silicic acid composition, comprising the step of combining a quantity of the present stabilized silicic acid solution with a quantity of water, such as plain tap water, e.g., in a ratio within the range of 1:1 to 1:50000, 1:2 to 1:1000 or 1:5 to 1:500, depending on the envisaged application. Yet a further aspect of the invention concerns a dilute and/or ready-to-use composition obtainable by said method.

### EXAMPLES

### General Information

All percentage (%) values of materials are expressed as weight portions (% w/w). The term “room temperature” (r.t.) relates to a temperature range of 20-25° C. The mixing speed is expressed as the number of revolutions per minute (r.p.m.) of the stirring element.

The starting raw materials were purchased from the following suppliers:
(1) 1,2-propylene glycol (3), Emprove® Essential, Ph.Eur.; pharma grade; Merck KGaA, Darmstadt (DE);
(2) polyethylene glycol 400 (4; PEG 400), Emprove® Essential, Ph.Eur.; pharma grade; Merck KgaA, Darmstadt (DE);
(3) polyethylene glycol 1000 (4; PEG 1000), for synthesis; Merck KgaA, Darmstadt (DE);
(4) sorbitol, anhydrous; Acros Organics, Geel (BE);
(5) sorbitol, 70% w/w solution; Carlo Erba, Milan (IT);
(6) PQ sodium silicate solution of type N® clear; composition: 8.9% w/w Na₂O, 28.7% SiO₂, 37.60% w/w total solids; PQ Corporation (US);
(7) potassium silicate solution of type KASIL®-6; composition: 12.7% w/w K₂O, 26.5% SiO₂, 39.21.0% w/w total solids; PQ Corporation (US);
(8) amino acids food grade, 98.5-101.5%; Gonmisol (ES); such as, glycine (Gly), L-leucine (Leu), L-proline (Pro), L-serine (Ser), L-threonine (Thr), L-aspartic acid (Asp), L-glutamic acid (Glu), L-phenylalanine (Phe), L-tyrosine (Tyr), L-tryptophan (Trp), L-histidine (His), L-arginine (Arg), L-glutamine (Glu), L-ornithine hydrochloride (Orn·HCl), L-lysine hydrochloride (Lys·HCl), L-methionine (Met), L-cysteine hydrochloride monohydrate (Cys·HCl·H₂O), L-citrulline DL-malate (Cit·Mal) and beta-alanine (βAl).
(9) sulfuric acid 95-97% for analysis Emsure® ISO; Merck KgaA, Darmstadt (DE);

All other starting raw materials and laboratory consumables were purchased from local suppliers.

### Example 1 (Comparative). Preparation of a Control Composition of Silicic Acid Based on Glycine (Gly), Glycerol, and Hydrochloric Acid (HCl) with 0.6% w/w Silicon (Si)

### Starting Materials (for 100 g Solution):

| | |
|---|---|
| (1) | 10.00% w/w (10.00 g) glycine (Gly); n(Gly) = 0.1332 mol; |
| (2) | 40.00% w/w (40.00 g) glycerol; |
| (3) | 30.74% w/w (30.74 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 14.41% w/w (14.41 g) hydrochloric acid, 37%; 5.33 g HCl, n(HCl) = 0.1462 mol. |

Total: 100% w/w (100.00 g)

Preparation: Glycine was dissolved in a mixture of purified water (10.00 g) and glycerol by stirring at r.t. during 5 min. Separately, hydrochloric acid (37%) was added dropwise to purified water (5.74 g) with external cooling at 15-20° C. for 5 min. and thus obtained diluted hydrochloric acid solution was added dropwise to the above-mentioned glycine solution at 15-20° C. during 5 min. Separately, potassium silicate solution KASIL®-6 was added to purified water (15.00 g) and shortly (1 min) homogenised by stirring. Thus obtained diluted potassium silicate solution was added dropwise to the above-mentioned solution of glycine hydrochloride-hydrochloric acid in aqueous-glycerol solution at 12-17° C. during 25-30 min.

Thus obtained product was in the form of an odourless, colourless, clear, slightly viscous solution; pH=0.80/20° C.; the composition of the product is as follows:
(1) silicic acid; equivalent to 0.60% w/w of silicon (Si);
(2) 14.86% w/w glycine hydrochloride;
(3) 0.98% w/w potassium chloride (KCl);
(4) 40.00% w/w glycerol; and
(5) ad 100% w/w purified water.

### Example 2 (Comparative). Preparation of a Control Composition of Silicic Acid Based on Glycine (Gly), Sorbitol, and Hydrochloric Acid (HCl) with 0.6% w/w Silicon (Si)

### Starting Materials (for 100 g Solution):

| | |
|---|---|
| (1) | 10.00% w/w (10.00 g) glycine (Gly); n(Gly) = 0.1332 mol; |
| (2) | 57.14% w/w (40.00 g) sorbitol, 70% w/w solution; |
| (3) | 13.60% w/w (13.60 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 14.41% w/w (14.41 g) hydrochloric acid, 37%; 5.33 g HCl, n(HCl) = 0.1462 mol. |

Total: 100% w/w (100.00 g)

Preparation: Glycine was suspended in sorbitol, 70% solution, by stirring at r.t. during 5 min. Separately, hydrochloric acid (37%) was added dropwise to purified water (3.60 g) with external cooling at 15-20° C. during 5 min., and thus obtained diluted hydrochloric acid solution was added dropwise to the above-mentioned glycine solution at 15-20° C. during 5 min. Separately, potassium silicate solution KASIL®-6 was added to purified water (10.00 g) and shortly (1 min) homogenised by stirring. Thus obtained diluted potassium silicate solution was added dropwise to above-mentioned solution of glycine hydrochloride-hydrochloric acid in aqueous-sorbitol solution at 12-17° C. during 25-30 min.

Thus obtained product was in the form of an odourless, colourless, clear, slightly viscous solution; pH=0.70/20° C.; the composition of the product is as follows:
(1) silicic acid; equivalent to 0.60% w/w of silicon (Si);
(2) 14.86% w/w glycine hydrochloride;
(3) 0.98% w/w potassium chloride (KCl);
(4) 40.00% w/w sorbitol; and
(5) ad 100% w/w purified water.

### Example 3 (Comparative). Preparation of a Control Composition of Silicic Acid Based on Glycine (Gly), Glycerol, and Phosphoric Acid (H₃PO₄) with 0.6% w/w Silicon (Si)

### Starting Materials (for 100 g Solution):

| | |
|---|---|
| (1) | 5.00% w/w (5.00 g) glycine (Gly); n(Gly) = 0.0666 mol; |
| (2) | 40.00% w/w (40.00 g) glycerol; |
| (3) | 40.72% w/w (40.72 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 9.43% w/w (9.43 g) phosphoric acid, 85%; 8.02 g H₃PO₄, n(H₃PO₄) = 0.0818 mol. |

Total: 100% w/w (100.00 g)

Preparation: Glycine was suspended in a mixture of purified water (10.00 g) and glycerol by stirring at r.t. during 5 min. Separately, phosphoric acid (85%) was added dropwise to purified water (10.72 g) with external cooling at 15-20° C. for 5 min. and thus obtained diluted phosphoric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine dihydrogenphosphate-phosphoric acid in aqueous-glycerol solution at 12-17° C. during 25-30 min.

Thus obtained product was in the form of an odourless, colourless, clear, slightly viscous solution; pH=2.23/20° C.; the composition of the product is as follows:
(1) silicic acid; equivalent to 0.60% w/w of silicon (Si);
(2) 11.53% w/w glycine dihydrogenphosphate;
(3) 1.78% w/w potassium dihydrogenphosphate (KH₂PO₄);
(4) 40.00% w/w glycerol; and
(5) ad 100% w/w purified water.

### Example 4 (Comparative). Preparation of a Control Composition of Silicic Acid Based on Glycine (Gly), Sorbitol, and Phosphoric Acid (H₃PO₄) with 0.6% w/w Silicon (Si)

### Starting Materials (for 100 g Solution):

| | |
|---|---|
| (1) | 5.00% w/w (5.00 g) glycine (Gly); n(Gly) = 0.0666 mol; |
| (2) | 40.00% w/w (40.00 g) sorbitol, anhydrous; |
| (3) | 40.72% w/w (40.72 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 9.43% w/w (9.43 g) phosphoric acid, 85%; 8.02 g H₃PO₄, n(H₃PO₄) = 0.0818 mol. |

Total: 100% w/w (100.00 g)

Preparation: Glycine was dissolved in a mixture of purified water (10.00 g) and sorbitol by stirring at r.t. for 5 min. Separately, phosphoric acid (85%) was added dropwise to purified water (10.72 g) with external cooling at 15-20° C. during 5 min. and thus obtained diluted phosphoric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine dihydrogenphosphate-phosphoric acid in aqueous-sorbitol solution at 12-17° C. during 25-30 min.

Thus obtained product was in the form of an odourless, colourless, clear, slightly viscous solution; pH=2.01/20° C.; the composition of the product is as follows:
(1) silicic acid; equivalent to 0.60% w/w of silicon (Si);
(2) 11.53% w/w glycine dihydrogenphosphate;
(3) 1.78% w/w potassium dihydrogenphosphate (KH₂PO₄);
(4) 40.00% w/w sorbitol; and
(5) ad 100% w/w purified water.

### Example 5 (Comparative). Preparation of a Control Composition of Silicic Acid Based on Glycine (Gly), Glycerol, and Sulfuric Acid (H₂SO₄) with 0.6% w/w Silicon (Si)

### Starting Materials (for 100 g Solution):

| | |
|---|---|
| (1) | 5.00% w/w (5.00 g) glycine (Gly); n(Gly) = 0.0666 mol; |
| (2) | 40.00% w/w (40.00 g) glycerol; |
| (3) | 42.01% w/w (42.01 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 8.14% w/w (8.14 g) sulfuric acid, 96%; 7.81 g H₂SO₄, n(H₂SO₄) = 0.0818 mol. |

Total: 100% w/w (100.00 g)

Preparation: Glycine was dissolved in a mixture of purified water (10.00 g) and glycerol by stirring at r.t. during 5 min. Separately, sulfuric acid (96%) was added dropwise to purified water (12.01 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine dihydrogensulfate-sulfuric acid in aqueous-glycerol solution at 12-17° C. during 25-30 min.

Thus obtained product was in the form of an odourless, colourless, clear, slightly viscous solution; pH=2.01/20° C.; the composition of the product is as follows:
(1) silicic acid; equivalent to 0.60% w/w of silicon (Si);
(2) 11.53% w/w glycine dihydrogensulfate (1);
(3) 1.78% w/w potassium dihydrogensulfate (KHSO₄);
(4) 40.00% w/w glycerol; and
(5) ad 100% w/w purified water.

### Example 6 (Comparative). Preparation of a Control Composition of Silicic Acid Based on Glycine (Gly), Sorbitol, and Sulfuric Acid (H₂SO₄) with 0.6% w/w Silicon (Si)

### Starting Materials (for 100 g Solution):

| | |
|---|---|
| (1) | 5.00% w/w (5.00 g) glycine (Gly); n(Gly) = 0.0666 mol; |
| (2) | 57.14% w/w (57.14 g) sorbitol, 70% w/w solution; |
| (3) | 24.87% w/w (24.87 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 8.14% w/w (8.14 g) sulfuric acid, 96%; 7.81 g H₂SO₄, n(H₂SO₄) = 0.0818 mol. |

Total: 100% w/w (100.00 g)

Preparation: Glycine was suspended in a mixture of purified water (4.87 g) and sorbitol, 70% solution, by stirring at r.t. during 5 min. Separately, sulfuric acid (96%) was added dropwise to purified water (10.00 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (10.00 g) and shortly (1 min) homogenised by stirring. Thus obtained diluted potassium silicate solution was added dropwise to the above-mentioned solution of glycine dihydrogensulfate-sulfuric acid in aqueous-sorbitol solution at 12-17° C. during 25-30 min.

Thus obtained product was in the form of an odourless, colourless, clear, slightly viscous solution; pH=2.01/20° C.; the composition of the product is as follows:
(1) silicic acid; equivalent to 0.60% w/w of silicon (Si);
(2) 11.53% w/w glycine dihydrogensulfate (1);
(3) 1.78% w/w potassium dihydrogensulfate (KHSO₄);
(4) 40.00% w/w sorbitol; and
(5) ad 100% w/w purified water.

### Example 7. A Preparation of the Composition from the Present Invention with 0.6% w/w Silicon (Si) Based on Glycine (Gly) and 1,2-Propylene Glycol (3)

### Starting Materials (for 100 g Solution):

| | |
|---|---|
| (1) | 1.61% w/w (1.61 g) glycine (Gly); n(Gly) = 0.02145 mol; |
| (2) | 40.00% w/w (40.00 g) 1,2-propylene glycol (3); |
| (3) | 50.01% w/w (50.01 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 3.53% w/w (3.53 g) sulfuric acid, 96%; 3.39 g H₂SO₄, n(H₂SO₄) = 0.03455 mol. |

Total: 100% w/w (100.00 g)

Preparation: Glycine was dissolved in a mixture of purified water (20.00 g) and 1,2-propylene glycol by stirring at r.t. during 5 min. Separately, sulfuric acid was added dropwise to purified water (10.01 g) with external cooling at 15-20° C. for 5 min. and thus obtained diluted sulfuric acid solution was added dropwise to above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=0.98/22° C.; the composition of the product is as follows:
(1) silicic acid; equivalent to 0.60% w/w of silicon (Si);
(2) 3.71% w/w glycine hydrogensulfate (1);
(5) 40.00% w/w 1,2-propylene glycol (3); and
(6) ad 100% w/w purified water.

### Example 8. Preparation of the Composition from the Present Invention with 0.6% w/w Silicon (Si) Based on Glycine (Gly) and Polyethylene Glycol 400 (4; PEG 400)

### Starting Materials (for 100 g Solution):

| | |
|---|---|
| (1) | 1.61% w/w (1.61 g) glycine (Gly); n(Gly) = 0.02145 mol; |
| (2) | 40.00% w/w (40.00 g) polyethylene glycol 400 (4; PEG 400); |
| (3) | 50.01% w/w (50.01 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 3.53% w/w (3.53 g) sulfuric acid, 96%; 3.39 g H₂SO₄, n(H₂SO₄) = 0.03455 mol. |

Total: 100% w/w (100.00 g)

Preparation: Glycine was dissolved in a mixture of purified water (20.00 g) and polyethylene glycol 400 (4; PEG 400) by stirring at r.t. during 5 min. Separately, sulfuric acid was added dropwise to purified water (10.01 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-PEG solution at 15-20° C. during 20-30 min.

Thus obtained product, stabilised silicic acid solution according to the present invention, was in the form of an odourless, colourless, clear, slightly viscous solution; PH=1.13/20° C.; the composition of the product is as follows:
(1) silicic acid; equivalent to 0.60% w/w of silicon (Si);
(2) 3.71% w/w glycine hydrogensulfate (1);
(4) 1.78% w/w potassium hydrogensulfate (KHSO₄);
(5) 40.00% w/w polyethylene glycol 400 (4; PEG 400); and
(6) ad 100% w/w purified water.

### Example 9. Preparation of the Composition from the Present Invention Based on Glycine (Gly) with 0.01% w/w Silicon (Si)

### Starting Materials (for 100 g Solution):

| | |
|---|---|
| (1) | 0.054% w/w (0.054 g) glycine (Gly); n(Gly) = 0.00072 mol; |
| (2) | 15.000% w/w (15.000 g) 1,2-propylene glycol (3); |
| (3) | 15.000% w/w (15.000 g) polyethylene glycol 1000 (4; PEG 1000); |
| (3) | 69.765% w/w (69.765 g) purified water; |
| (4) | 0.081% w/w (0.081 g) potassium silicate solution, type KASIL ®-6; contains |
| | 12.7% w/w K₂O and 26.5% w/w SiO₂; equivalent to: 0.0103 g |
| | K₂O, n(K₂O) = 0.00011 mol; 0.0215 g SiO₂, n(SiO₂) = 0.00036 |
| | mol; |
| (5) | 0.100% w/w (0.100 g) sulfuric acid, 96%; 0.096 g H₂SO₄, n(H₂SO₄) = 0.00098 mol. |

Total: 100% w/w (100.00 g)

Preparation: Glycine was dissolved in a mixture of purified water (49.765 g), 1,2-propylene glycol (3), and polyethylene glycol 1000 (4; PEG 1000) by stirring at r.t. during 15 min. Separately, sulfuric acid was added dropwise to the above-mentioned glycine solution at 15-20° C. for 1 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol-PEG solution at 15-20° C. during 25-30 min.

Thus obtained product, stabilised silicic acid solution according to the present invention, was in the form of an odourless, colourless, clear, slightly viscous solution; pH=1.40/20° C.; the composition of the product is as follows:
(1) silicic acid; equivalent to 0.01% w/w of silicon (Si);
(2) 0.125% w/w glycine hydrogensulfate (1);
(3) 0.000% w/w potassium glycine sulfate (2b);
(4) 0.030% w/w potassium hydrogensulfate (KHSO₄);
(5) 15.00% w/w 1,2-propylene glycol (3);
(6) 15.00% w/w polyethylene glycol 1000 (4; PEG 1000); and
(7) ad 100% w/w purified water.

### Example 10. Preparation of the Composition from the Present Invention Based on Glycine (Gly) with 0.1% w/w Silicon (Si)

### Starting Materials (for 100 g Solution):

| | |
|---|---|
| (1) | 0.27% w/w (0.27 g) glycine (Gly); n(Gly) = 0.00360 mol; |
| (2) | 10.00% w/w (10.00 g) 1,2-propylene glycol (3); |
| (3) | 5.00% w/w (5.00 g) polyethylene glycol 400 (4; PEG 400); |
| (4) | 83.39% w/w (83.39 g) purified water; |
| (5) | 0.75% w/w (0.75 g) sodium silicate solution, type N ® Clear; contains 8.9% |
| | w/w Na₂O and 28.7% w/w SiO₂; equivalent to: 0.0668 g Na₂O, |
| | n(Na₂O) = 0.00108 mol; 0.2153 g SiO₂, n(SiO₂) = 0.00358 mol; |
| (6) | 0.59% w/w (0.59 g) sulfuric acid, 96%; 0.566 g H₂SO₄, n(H₂SO₄) = 0.00577 mol. |

Total: 100% w/w (100.00 g)

Preparation: Glycine was dissolved in a mixture of purified water (50.00 g), 1,2-propylene glycol, and PEG 400 by stirring at r.t. during 5 min. Separately, sulfuric acid was added dropwise to purified water (13.39 g) with external cooling at 15-20° C. for 5 min. and thus obtained diluted sulfuric acid solution was added dropwise to the above-mentioned glycine solution at 15-20° C. during 5 min. Separately, sodium silicate solution N® Clear was added to purified water (20.00 g) and shortly (1 min) homogenised by stirring. Thus obtained diluted sodium silicate solution was added dropwise to the above-mentioned solution of glycine hydrogensulfate (1)-sulfuric acid in aqueous-1,2-propylene glycol-PEG solution at 15-20° C. during 20-30 min.

Thus obtained product, stabilised silicic acid solution according to the present invention, was in the form of an odourless, colourless, clear, slightly viscous solution; pH=1.30/20° C.; the composition of the product is as follows:
(1) silicic acid; equivalent to 0.10% w/w of silicon (Si);
(2) 0.62% w/w glycine hydrogensulfate (1);
(3) 0.00% w/w sodium glycine sulfate (2a);
(4) 0.26% w/w sodium hydrogensulfate (NaHSO₄);
(5) 10.00% w/w 1,2-propylene glycol (3);
(6) 5.00% w/w polyethylene glycol 400 (4; PEG 400); and
(7) ad 100% w/w purified water.

### Example 11. Preparation of the Composition from the Present Invention Based on Glycine (Gly) with 0.4% w/w Silicon (Si)

### Starting Materials (for 100 g Solution):

| | |
|---|---|
| (1) | 1.07% w/w (1.07 g) glycine (Gly); n(Gly) = 0.01425 mol; |
| (2) | 40.00% w/w (40.00 g) 1,2-propylene glycol (3); |
| (3) | 53.36% w/w (53.36 g) purified water; |
| (4) | 3.23% w/w (3.23 g) potassium silicate solution, type KASIL ®-6; contains |
| | 12.7% w/w K₂O and 26.5% w/w SiO₂; equivalent to: 0.410 g |
| | K₂O, n(K₂O) = 0.00435 mol; 0.856 g SiO₂, n(SiO₂) = 0.01425 |
| | mol; |
| (5) | 2.34% w/w (2.34 g) sulfuric acid, 96%; 2.25 g H₂SO₄, n(H₂SO₄) = 0.02294 mol. |

Total: 100% w/w (100.00 g)

Preparation: Glycine was dissolved in a mixture of purified water (20.00 g) and 1,2-propylene glycol by stirring at r.t. during 5 min. Separately, sulfuric acid was added dropwise to purified water (13.36 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 15-20° C. during 25-30 min.

Thus obtained product, stabilised silicic acid solution according to the present invention, was in the form of an odourless, colourless, clear, slightly viscous solution; pH=1.07/20° C.; the composition of the product is as follows:
(1) silicic acid; equivalent to 0.40% w/w of silicon (Si);
(2) 2.47% w/w glycine hydrogensulfate (1);
(3) 0.00% w/w potassium glycine sulfate (2b);
(4) 1.18% w/w potassium hydrogensulfate (KHSO₄);
(5) 40.00% w/w 1,2-propylene glycol (3); and
(6) ad 100% w/w purified water.

### Example 12. Preparation of the Composition from the Present Invention Based on Glycine (Gly) with 0.5% w/w Silicon (Si)

### Starting Materials (for 100 g Solution):

| | |
|---|---|
| (1) | 1.34% w/w (1.34 g) glycine (Gly); n(Gly) = 0.01785 mol; |
| (2) | 40.00% w/w (40.00 g) 1,2-propylene glycol (3); |
| (3) | 52.59% w/w (52.59 g) purified water; |
| (4) | 4.04% w/w (4.04 g) potassium silicate solution, type KASIL ®-6; contains |
| | 12.7% w/w K₂O and 26.5% w/w SiO₂; equivalent to: 0.513 g |
| | K₂O, n(K₂O) = 0.00545 mol; 1.071 g SiO₂, n(SiO₂) = 0.01782 mol; |
| (5) | 2.03% w/w (2.03 g) sulfuric acid, 96%; 1.95 g H₂SO₄, n(H₂SO₄) = 0.01987 mol. |

Total: 100% w/w (100.00 g)

Preparation: Glycine was dissolved in a mixture of purified water (20.00 g) and 1,2-propylene glycol by stirring at r.t. during 5 min. Separately, sulfuric acid was added dropwise to purified water (12.59 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 15-20° C. during 25-30 min.

Thus obtained product, stabilised silicic acid solution according to the present invention, was in the form of an odourless, colourless, clear, slightly viscous solution; pH=1.70/20° C.; the composition of the product is as follows:
(1) silicic acid; equivalent to 0.40% w/w of silicon (Si);
(2) 1.55% w/w glycine hydrogensulfate (1);
(3) 1.88% w/w potassium glycine sulfate (2b);
(4) 0.28% w/w potassium hydrogensulfate (KHSO₄);
(5) 40.00% w/w 1,2-propylene glycol (3); and
(6) ad 100% w/w purified water.

### Example 13. Preparation of the Composition from the Present Invention Based on Glycine (Gly) with 0.6% w/w Silicon (Si)

### Starting Materials (for 100 g Solution):

| | |
|---|---|
| (1) | 1.61% w/w (1.61 g) glycine (Gly); n(Gly) = 0.02145 mol; |
| (2) | 50.00% w/w (50.00 g) 1,2-propylene glycol (3); |
| (3) | 40.56% w/w (40.56 g) purified water; |
| (4) | 4.85% w/w (4.85 g) potassium silicate solution, type KASIL ®-6; contains |
| | 12.7% w/w K₂O and 26.5% w/w SiO₂; equivalent to: 0.616 g |
| | K₂O, n(K₂O) = 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02917 mol. |

Total: 100% w/w (100.00 g)

Preparation: Glycine was dissolved in a mixture of purified water (15.00 g) and 1,2-propylene glycol by stirring at r.t. during 5 min. Separately, sulfuric acid was added dropwise to purified water (10.56 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (15.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.43/21° C.; the composition of the product is:
(1) silicic acid; equivalent to 0.60% w/w of silicon (Si);
(2) 2.79% w/w glycine hydrogensulfate (1);
(3) 1.13% w/w potassium glycine sulfate (2b);
(4) 1.05% w/w potassium hydrogensulfate (KHSO₄);
(5) 50.00% w/w 1,2-propylene glycol (3); and
(6) ad 100% w/w purified water.

### Example 14. Preparation of the Composition from the Present Invention Based on Glycine (Gly) with 1.0% w/w Silicon (Si)

### Starting Materials (for 100 g Solution):

| | |
|---|---|
| (1) | 2.68% w/w (2.68 g) glycine (Gly); n(Gly) = 0.0357 mol; |
| (2) | 40.00% w/w (40.00 g) polyethylene glycol 400 (4; PEG 400); |
| (3) | 44.28% w/w (44.28 g) purified water; |
| (4) | 8.08% w/w (8.08 g) potassium silicate solution, type KASIL ®-6; contains |
| | 12.7% w/w K₂O and 26.5% w/w SiO₂; equivalent to: 1.026 g |
| | K₂O, n(K₂O) = 0.01089 mol; 2.141 g SiO₂, n(SiO₂) = 0.03563 mol; |
| (5) | 4.96% w/w (4.96 g) sulfuric acid, 96%; 4.76 g H₂SO₄, n(H₂SO₄) = 0.04855 mol. |

Total: 100% w/w (100.00 g)

Preparation: Glycine was dissolved in a mixture of purified water (15.00 g) and polyethylene glycol 400 (4; PEG 400) by stirring at r.t. during 5 min. Separately, sulfuric acid was added dropwise to purified water (14.28 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (15.00 g) and shortly (1 min) homogenised by stirring. Thus obtained diluted potassium silicate solution was added dropwise to the above-mentioned solution of glycine hydrogensulfate (1)-sulfuric acid in aqueous-PEG solution at 15-20° C. during 25-30 min.

Thus obtained product, stabilised silicic acid solution according to the present invention, was in the form of an odourless, colourless, clear, slightly viscous solution; pH=1.47/20° C.; the composition of the product is as follows:
(1) silicic acid; equivalent to 1.00% w/w of silicon (Si);
(2) 6.18% w/w glycine hydrogensulfate (1);
(3) 1.89% w/w potassium glycine sulfate (2b);
(4) 1.75% w/w potassium hydrogensulfate (KHSO₄);
(5) 40.00% w/w polyethylene glycol 400 (4; PEG 400); and
(6) ad 100% w/w purified water.

### Example 15. Preparation of the Composition from the Present Invention Based on Glycine (Gly) with 0.6% w/w Silicon (Si)

### Starting Materials (for 100 g Solution):

| | |
|---|---|
| (1) | 1.61% w/w (1.61 g) glycine (Gly); n(Gly) = 0.02145 mol; |
| (2) | 40.00% w/w (40.00 g) 1,2-propylene glycol (3); |
| (3) | 51.65% w/w (51.65 g) purified water; |
| (4) | 4.85% w/w (4.85 g) potassium silicate solution, type KASIL ®-6; contains |
| | 12.7% w/w K₂O and 26.5% w/w SiO₂; equivalent to: 0.616 g |
| | K₂O, n(K₂O) = 0.00654 mol; 1.285 g SiO₂, n(SiO₂) = 0.02139 mol; |
| (5) | 1.89% w/w (1.89 g) sulfuric acid, 96%; 1.81 g H₂SO₄, n(H₂SO₄) = 0.0185 mol. |

Total: 100% w/w (100.00 g)

Preparation: Glycine was dissolved in a mixture of purified water (20.00 g) and 1,2-propylene glycol by stirring at r.t. during 5 min. Separately, sulfuric acid was added dropwise to purified water (10.65 g) with external cooling at 10-15° C. for 5 min. and thus obtained diluted sulfuric acid solution was added dropwise to the above-mentioned glycine solution at 10-17° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=2.20/20° C.; the composition of the product is:
(1) silicic acid; equivalent to 0.60% w/w of silicon (Si);
(2) 0.93% w/w glycine hydrogensulfate (1);
(3) 2.76% w/w potassium glycine sulfate (2b);
(4) 0.00% w/w potassium hydrogensulfate (KHSO₄);
(5) 40.00% w/w 1,2-propylene glycol (3); and
(6) ad 100% w/w purified water.

### Example 16. Preparation of the Composition from the Present Invention Based on Glycine (Gly) with 0.6% w/w Silicon (Si)

### Starting Materials (for 100 g Solution):

| | |
|---|---|
| (1) | 1.61% w/w (1.61 g) glycine (Gly); n(Gly) = 0.02145 mol; |
| (2) | 40.00% w/w (40.00 g) 1,2-propylene glycol (3); |
| (3) | 51.65% w/w (51.65 g) purified water; |
| (4) | 4.85% w/w (4.85 g) potassium silicate solution, type KASIL ®-6; contains |
| | 12.7% w/w K₂O and 26.5% w/w SiO₂; equivalent to: 0.616 g |
| | K₂O, n(K₂O)= 0.00654 mol; 1.285 g SiO₂, n(SiO₂) = 0.02139 mol; |
| (5) | 2.43% w/w (2.43 g) sulfuric acid, 96%; 2.33 g H₂SO₄, n(H₂SO₄) = 0.023785 mol |

Total: 100% w/w (100.00 g)

Preparation: Glycine was dissolved in a mixture of purified water (20.00 g) and 1,2-propylene glycol by stirring at r.t. during 5 min. Separately, sulfuric acid was added dropwise to purified water (10.65 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 13-17° C. during 25-30 min.

Thus obtained product, stabilised silicic acid solution according to the present invention, was in the form of an odourless, colourless, clear, slightly viscous solution; pH=1.65/20° C.; the composition of the product is:
(1) silicic acid; equivalent to 0.60% w/w of silicon (Si);
(2) 1.85% w/w glycine hydrogensulfate (1);
(3) 2.27% w/w potassium glycine sulfate (2b);
(4) 0.32% w/w potassium hydrogensulfate (KHSO₄);
(5) 40.00% w/w 1,2-propylene glycol (3); and
(6) ad 100% w/w purified water.

### Example 17. Preparation of the Composition from the Present Invention Based on Glycine (Gly) with 0.6% w/w Silicon (Si)

### Starting Materials (for 100 g Solution):

| | |
|---|---|
| (1) | 1.61% w/w (1.61 g) glycine (Gly); n(Gly) = 0.02145 mol; |
| (2) | 40.00% w/w (40.00 g) 1,2-propylene glycol (3); |
| (3) | 50.56% w/w (50.56 g) purified water; |
| (4) | 4.85% w/w (4.85 g) potassium silicate solution, type KASIL ®-6; contains |
| | 12.7% w/w K₂O and 26.5% w/w SiO₂; equivalent to: 0.616 g |
| | K₂O, n(K₂O) = 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02917 mol |

Total: 100% w/w (100.00 g)

Preparation: Glycine was dissolved in a mixture of purified water (20.00 g) and 1,2-propylene glycol by stirring at r.t. during 5 min. Separately, sulfuric acid was added dropwise to purified water (10.56 g) with external cooling at 15-20° C. for 5 min. and thus obtained diluted sulfuric acid solution was added dropwise to the above-mentioned glycine solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of glycine hydrogensulfate (1)-sulfuric acid in aqueous-1,2-propylene glycol solution at 13-17° C. during 25-30 min.

Thus obtained product, stabilised silicic acid solution according to the present invention, was in the form of an odourless, colourless, clear, slightly viscous solution; pH=1.65/20° C.; the composition of the product is:
(1) silicic acid; equivalent to 0.60% w/w of silicon (Si);
(2) 2.79% w/w glycine hydrogensulfate (1);
(3) 1.13% w/w potassium glycine sulfate (2b);
(4) 1.05% w/w potassium hydrogensulfate (KHSO₄);
(5) 40.00% w/w 1,2-propylene glycol (3); and
(6) ad 100% w/w purified water.

### Example 18. Preparation of the Composition from the Present Invention with Analogues of Eighteen Different Amino Acids with 0.6%, 0.3% and 0.25% w/w Silicon (Si)

### Starting Materials (for 100 g Solution):

| | |
|---|---|
| (1) | m₁ g (n-0.04278 mol; 2 mol. equiv. to Si) amino acid [L-leucine (Leu), L- |
| | proline (Pro), L-serine (Ser), L-threonine (Thr), L-aspartic acid (Asp), L- |
| | glutamic acid (Glu), L-phenylalanine (Phe), L-tyrosine (Tyr), L-tryptophan |
| | (Trp), L-histidine (His), L-arginine (Arg), L-glutamine (Gln), L-ornithine |
| | hydrochloride (Orn•HCl), L-lysine hydrochloride (Lys•HCl), L-methionine |
| | (Met), L-cysteine hydrochloride monohydrate (Cys•HCl•H₂O), L-citrulline DL- |
| | malate (Cit•Mal), or beta-alanine (βAl)];^{a} |
| (2) | 40.00% w/w (40.00 g) 1,2-propylene glycol (3); |
| (3) | m₂ g purified water; |
| (4) | m₃ g sulfuric acid (H₂SO₄; 96% w/w);^{b} |
| (5) | m₄ g purified water; |
| (6) | 4.85% w/w (4.85 g) potassium silicate solution, type KASIL ®-6; for runs 6-10, |
| | 12-13 and 15-23; contains 12.7% w/w K₂O and 26.5% w/w |
| | SiO₂; equivalent to: 0.616 g K₂O, n(K₂O) = 0.00654 mol; 1.285 g |
| | SiO₂, n(SiO₂) = 0.02139 mol; equivalent to 0.60% w/w Si in the final product.^{c} |

Total: 100% w/w (100.00 g)

The quantities of m₁-m₄ used in these experiments are as follows:

| Run | Amino acid (AA) | m₁ g^{a} | m₂ g | m₃ g^{b} | m₄ g |
|---|---|---|---|---|---|
| 1 | Leu | 2.82 | 30.00 | 2.98 | 19.35 |
| 2 | Pro | 2.47 | 30.00 | 2.98 | 19.70 |
| 3 | Ser | 2.25 | 30.00 | 2.98 | 19.92 |
| 4 | Thr | 2.55 | 30.00 | 2.98 | 19.62 |
| 5 | Asp | 2.85 | 30.00 | 2.98 | 19.32 |
| 6 | Glu | 3.16 | 30.00 | 2.98 | 19.01 |
| 7 | Phe | 3.54 | 30.00 | 2.98 | 18.63 |
| 8 | Tyr | 3.89 | 30.00 | 2.98 | 18.28 |
| 9 | Trp | 4.38 | 30.00 | 2.98 | 17.79 |
| 10 | His | 3.33 | 25.00 | 5.17^{d} | 21.65 |
| 11 | Arg | 3.74 | 25.00 | 5.17^{d} | 21.24 |
| 12 | Gln | 3.13 | 30.00 | 2.98 | 19.04 |
| 13 | Orn•HCl | 3.62 | 30.00 | 2.98^{e} | 18.55 |
| 14 | Lys•HCl | 3.92 | 30.00 | 2.98^{e} | 18.25 |
| 15 | Met | 3.20 | 30.00 | 2.98 | 18.97 |
| 16 | Cys•HCl•H₂O | 3.77 | 30.00 | 1.34^{f} | 20.04 |
| 17 | Cit DL-malate | 5.20 | 25.00 | 2.98^{e} | 21.97 |
| 18 | βAl | 1.91 | 30.00 | 2.98 | 20.26 |

^{a}The quantity of each amino acid (AA) was equimolar to silicon: n(Si):n(AA) = 1:1.
^{b}The quantity of sulfuric acid employed equals the sum of: (a) the amount of substance of K⁺ (from K₂O) and (b) 75% of the amount of substance of the corresponding amino acid (AA) unless otherwise noted.
^{c} The quantity of potassium silicate solution (KASIL ® 6) corresponds to the content of silicon (Si) equal to 0.60% w/w Si in the final products.
^{d}The quantity of sulfuric acid employed in these cases equals the sum of: (i) the amount of substance of the corresponding AA required for neutralization of side-chain basic imidazole (His) or guanidine (Arg) groups; (ii) 75% of the amount of substance of the corresponding AA required for partial neutralization of
remained a-amino group; and (iii) the amount of substance of K⁺ (from K₂O) required for the conversion of all K₂O (from KASIL ® 6) to KHSO₄.
^{e}L-Ornithine and L-lysine are only available as hydrochloride salts, while L-citrulline is available as DL-malate salt (2:1). Therefore, the quantity of sulfuric acid in these cases was equal to the sum of: (i) the amount of substance of K⁺ (from K₂O); and (ii) 75% of the amount of substance of the corresponding amino acid (AA).
^{f}L-Cysteine is available as hydrochloride monohydrate salt. In this case, the quantity of sulfuric acid employed is equal to the amount of substance of K⁺ (from K₂O).

Preparation: An amino acid (AA), according to column m₁ was added to 1,2-propylene glycol by stirring at r.t. during 5 min. Separately, sulfuric acid (m₃) was added dropwise to purified water (m₂) with stirring for 1 minute and thus obtained diluted sulfuric acid solution was added dropwise to the above-mentioned AA solution at 15-20° C. during 15 min. Separately, potassium silicate solution KASIL®-6 (4.85 g) was added to purified water (m₄) and homogenised by stirring for 5 min. Thus obtained diluted potassium silicate solution was added dropwise to the above-mentioned solution of amino acid (AA)-sulfuric acid in aqueous-1,2-propylene glycol solution at 13-17° C. during 25-30 min.

Thus obtained products, stabilised silicic acid solution according to the present invention, displayed the following key characteristics:

| | | Key characteristics of products from runs 1-18 | | | | | |
|---|---|---|---|---|---|---|---|
| | | n(Si):n (AA) = 1:1, n/n | | | | | |
| | | | | | Density | Si | AA |
| | Amino Acid | | | pH at | [g/cm³] | content | content |
| Run | (AA) | Appearance | Odour | 22° C. | at 20° C. | [%, w/w] | [%, w/w] |
| 1 | Leu | A | FO | 1.24 | 1.053 | 0.60 | 2.82 |
| 2 | Pro | A | N | 1.10 | 1.062 | 0.60 | 2.47 |
| 3 | Ser | A | FO | 1.15 | 1.064 | 0.60 | 2.25 |
| 4 | Thr | A | N | 1.14 | 1.064 | 0.60 | 2.55 |
| 5 | Asp | A | N | 1.05 | 1.066 | 0.60 | 2.80^{a} |
| 6 | Glu | A | N | 1.20 | 1.063 | 0.60 | 3.16 |
| 7 | Phe | A | FO | 1.28 | 1.057 | 0.60 | 3.54 |
| 8 | Tyr | A | FO | 0.88 | n.d. | 0.60 | 2.33^{b} |
| 9 | Trp | B | N | 1.28 | 1.063 | 0.60 | 4.38 |
| 10 | His | A | FO | 0.81 | 1.076 | 0.60 | 3.33 |
| 11 | Arg | A | FO | 0.82 | 1.071 | 0.60 | 3.74 |
| 12 | Gln | A | FO | 1.20 | 1.061 | 0.60 | 3.13 |
| 13 | Orn•HCl | A | N | 1.12 | 1.063 | 0.60 | 2.83 |
| 14 | Lys•HCl | A | N | 1.09 | 1.063 | 0.60 | 3.13 |
| 15 | Met | A | FOS1 | 1.22 | 1.061 | 0.60 | 3.20 |
| 16 | Cys•HCl•H₂O | A | FOS2 | 1.22 | 1.055 | 0.60 | 2.59 |
| 17 | Cit DL-malate | A | FO | 1.25 | 1.069 | 0.60 | 3.75^{c} |
| 18 | βAl | A | N | 1.28 | 1.060 | 0.60 | 1.91 |

A = colourless, clear viscous liquid, approximate viscosity <1,2-propylene glycol (1,2-PG)
B = pale brownish, clear viscous liquid, approximate viscosity <1,2-propylene glycol (1,2-PG)
FO = faintly intensive, characteristic
N = odourless
FOS1 = mildly intensive, characteristic, sulphurous
FOS2 = faintly intensive, characteristic, sulphurous
^{a}This was an atypical preparation. The reaction product was not a clear solution but a suspension of a small amount of white crystalline powder in colourless, clear viscous supernatant. The precipitate was removed during the filtration of the reaction product and dried at 105° C. til the constant weight yielded about 50 mg (≈2%) of starting Asp or its hydrogensulfate salt. Obviously, Asp-hydrogensulfate at such a concentration level is not fully soluble in the 1,2-PG − water. The final product is a clear solution, but upon standing at room temperature is prone to further crystallisation of Asp-hydrogensulfate yielding a small amount of colourless crystalline precipitate, approximately 50 mg per 50 g (1%).
^{b}This was also an atypical preparation. The reaction product was not a clear solution but a suspension of a white crystalline powder in colourless, clear viscous supernatant. The precipitate was removed during the filtration of the reaction product and dried at 105° C. til the constant weight yielded 1.71 g (≈44%) of starting Tyr or its hydrogensulfate salt. In this manner, the liquid product (filtrate) contains only approx. 60% of starting Tyr and this product contains a proportionally lowered stoichiometric ratio of Si:Tyr ≈1:0.6. Additionally, a colourless crystalline precipitate appeared from a clear product solution upon standing at room temperature for a few days. The amount of this precipitate from the product is about 200 mg/50 g product (4%).
^{c}This product also contains 1.43 g (1.43%) DL-malic acid from L-citrulline DL-malate (2:1) precursor.

### Example 19: Preparation of the Composition from the Present Invention with Analogues of Eighteen Different Amino Acids with 0.6%, 0.3% and 0.25% w/w Silicon (Si)

### Starting Materials (for 100 g Solution):

| | |
|---|---|
| (1) | m₁ g (n = 0.04278 mol; 2 mol. equiv. to Si) amino acid [L-leucine (Leu), L- |
| | proline (Pro), L-serine (Ser), L-threonine (Thr), L-aspartic acid (Asp), L- |
| | glutamic acid (Glu), L-phenylalanine (Phe), L-tyrosine (Tyr), L-tryptophan |
| | (Trp), L-histidine (His), L-arginine (Arg), L-glutamine (Gln), L-ornithine |
| | hydrochloride (Orn•HCl), L-lysine hydrochloride (Lys•HCl), L-methionine |
| | (Met), L-cysteine hydrochloride monohydrate (Cys•HCl•H₂O), L-citrulline DL- |
| | malate (Cit Mal), or beta-alanine (βAl)];^{a} |
| (2) | 40.00% w/w (40.00 g) 1,2-propylene glycol (3); |
| (3) | m₂ g purified water; |
| (4) | m₃ g sulfuric acid (H2SO4, 96% w/w);b |
| (5) | m₄ g purified water; |
| (6) | 4.85% w/w (4.85 g) potassium silicate solution, type KASIL ®-6; for runs 24- |
| | 28, 29-30, and 32-39; contains 12.7% w/w K₂O and 26.5% w/w |
| | SiO₂; equivalent to: 0.616 g K₂O, n(K₂O) = 0.00654 mol; 1.285 g |
| | SiO₂, n(SiO₂) = 0.02139 mol; equivalent to 0.60% w/w Si in the |
| | final product;^{c} |
| | or; |
| | 2.43% w/w (2.43 g) for run 29; m(K₂O) = 0.30861 g; n(K₂O) = 0.003276 mol; |
| | m(SiO₂) = 0.64395 g; n(SiO₂) = 0.010717 mol; equivalent to |
| | 0.30% w/w Si in the final product;^{c} |
| | or; |
| | 2.02% w/w (2.02 g) for run 32; m(K₂O) = 0.25654 g; n(K₂O) = 0.002723 mol; |
| | m(SiO₂) = 0.5353 g; n(SiO₂) = 0.008909 mol; equivalent to |
| | 0.25% w/w Si in the final product.^{c} |

Total: 100% w/w (100.00 g)

The quantities of m₁-m₄ used in these experiments are as follows:

| Run | Amino acid (AA) | m₁ g^{a} | m₂ g | m₃ g^{b} | m₄ g |
|---|---|---|---|---|---|
| 19 | Leu | 5.63 | 25.00 | 4.62 | 19.90 |
| 20 | Pro | 4.94 | 25.00 | 4.62 | 20.59 |
| 21 | Ser | 4.51 | 25.00 | 4.62 | 21.02 |
| 22 | Thr | 5.11 | 25.00 | 4.62 | 20.43 |
| 23 | Asp | 5.70 | 25.00 | 4.62 | 19.83 |
| 24 | Asp | 2.85d | 25.00 | 2.31 | 27.41 |
| 25 | Glu | 6.31 | 25.00 | 4.62 | 19.22 |
| 26 | Phe | 7.09 | 25.00 | 4.62 | 18.44 |
| 27 | Tyr | 3.23 | 25.00 | 2.38 | 27.37 |
| 28 | Trp | 8.73 | 25.00 | 4.62 | 16.80 |
| 29 | His | 6.63 | 20.00 | 7.88^{e} | 20.64 |
| 30 | Arg | 7.44 | 20.00 | 7.88^{e} | 19.83 |
| 31 | Gln | 6.24 | 25.00 | 4.62 | 19.29 |
| 32 | Orn•HCl | 7.20 | 25.00 | 4.62^{f} | 18.33 |
| 33 | Lys•HCl | 7.80 | 25.00 | 4.62^{f} | 17.73 |
| 34 | Met | 6.38 | 25.00 | 4.62 | 19.15 |
| 35 | Cys•HCl•H₂O | 7.50 | 25.00 | 1.34^{g} | 21.31 |
| 36 | Cit DL-malate | 10.35 | 20.00 | 4.62^{f} | 20.18 |
| 37 | βAl | 3.812 | 25.00 | 4.62 | 21.72 |

^{a}The molar ratio of each amino acid (AA) to silicon was: n(Si):n(AA) = 1:2.
^{b}The quantity of sulfuric acid employed equals the sum of: (a) the amount of substance of K⁺ (from K₂O) and (b) 75% of the amount of substance of the corresponding amino acid (AA) unless otherwise noted; see runs 24, 27, and 35.
^{c} The quantity of potassium silicate solution (KASIL ® 6) corresponds to the content of silicon equal to 0.60% w/w Si in the final product unless otherwise noted (run 24 and run 27).
^{d} In run 24, the complex of Asp was prepared with 0.30% w/w Si.
^{e}The quantity of sulfuric acid employed in these cases equals to: (a) the amount of substance of the corresponding AA required for neutralization of side-chain basic imidazole (His) or amidine (Arg) groups, (b) 75% of the amount of substance of the corresponding AA required for partial neutralization of remained alpha-amino group; and (c) amount of substance of K⁺ from K₂O required for the conversion of all K₂O (from KASIL ® 6) to KHSO₄.
^{f}L-Ornithine, L-lysine are only available as hydrochloride salts, while L-citrulline is available only as DL-malate salt (2:1). Therefore, the quantity of sulfuric acid in these cases was equal to the sum of: (a) the amount of substance of K⁺ (from K₂O) and (b) 75% of the amount of substance of the corresponding amino acid (AA).
^{g}L-Cysteine is available as hydrochloride monohydrate salt. In this case, the quantity of sulfuric acid employed is equal to the amount of substance of K⁺ (from K₂O).

Preparation: An amino acid (AA), according to column m₁ was added to 1,2-propylene glycol by stirring at r.t. during 5 min. Separately, sulfuric acid (m₃) was added dropwise to purified water (m₂) with stirring for 1 minute and thus obtained diluted sulfuric acid solution was added dropwise to the above-mentioned AA solution at 15-20° C. during 15 min. Separately, potassium silicate solution KASIL®-6 (4.85 g) (2.43 g for experiment 29 and 2.02 g for experiment 32) was added to purified water (m₄) and homogenised by stirring for 5 min. Thus obtained diluted potassium silicate solution was added dropwise to the above-mentioned solution of amino acid (AA)-sulfuric acid in aqueous-1,2-propylene glycol solution at 13-17° C. during 25-30 min.

Thus obtained products, stabilised silicic acid solution according to the present invention, displayed the following key characteristics:

| | | Key characteristics of products from runs 19-37 of stoichiometry | | | | | |
|---|---|---|---|---|---|---|---|
| | | n(Si):n (AA) = 1:2, n/n | | | | | |
| | | | | | Density | Si | AA |
| | Amino Acid | | | pH at | [g/cm³] | content | content |
| Run | (AA) | Appearance | Odour | 22° C. | at 20° C. | [%, w/w] | [%, w/w] |
| 19 | Leu | A | FO | 1.17 | 1.071 | 0.60 | 5.63 |
| 20 | Pro | A | N | 1.12 | 1.071 | 0.60 | 4.94 |
| 21 | Ser | A | FO | 1.02 | 1.082 | 0.60 | 4.51 |
| 22 | Thr | A | N | 1.05 | 1.081 | 0.60 | 5.11 |
| 23 | Asp^{a} | S | N | n.d | n.d. | 0.60 | 5.70^{b} |
| 24 | Asp^{c} | A | N | 1.10 | 1.052 | 0.30 | 2.70^{d} |
| 25 | Glu | A | N | 1.04 | 1.084 | 0.60 | 6.31 |
| 26 | Phe | A | FO | 1.13 | 1.075 | 0.60 | 7.09 |
| 27 | Tyr^{e} | A | FO | 0.92 | 1.048 | 0.25 | 2.26^{f} |
| 28 | Trp | B | N | 1.14 | 1.081 | 0.60 | 8.73 |
| 29 | His | A | FC | 0.90 | 1.105 | 0.60 | 6.63 |
| 30 | Arg | A | FO | 0.80 | 1.104 | 0.60 | 7.44 |
| 31 | Gln | A | FO | 1.26 | 1.079 | 0.60 | 6.24 |
| 32 | Orn•HCl | A | FO | 0.85 | 1.086 | 0.60 | 5.64 |
| 33 | Lys•HCl | A | FO | 0.93 | 1.085 | 0.60 | 6.24 |
| 34 | Met | A | FOS1 | 1.17 | 1.078 | 0.60 | 6.38 |
| 35 | Cys•HCl•H₂O | A | FOS2 | 1.05 | 1.068 | 0.60 | 5.17 |
| 36 | Cit DL-malate^{c} | A | FO | 1.12 | 1.094 | 0.60 | 7.49^{g} |
| 37 | βAl | A | N / FO | 1.20 | 1.074 | 0.60 | 3.81 |

A = colourless, clear viscous liquid, approximate viscosity <1,2-propylene glycol (1,2-PG)
B = pale brownish, clear viscous liquid, approximate viscosity <1,2-propylene glycol (1,2-PG)
S = suspension; white crystalline powder suspended in a viscous clear supernatant
FO = faintly intensive, characteristic
N = odourless
FOS1 = mildly intensive, characteristic, sulphurous
FOS2 = faintly intensive, characteristic, sulphurous
n.d. = not determined
^{a}This was an atypical preparation. The reaction product was not a clear solution but a suspension of a white crystalline powder in colourless, clear viscous supernatant, which did not fully dissolve until the end of the preparation procedure. The precipitate was removed during the filtration of the reaction product and dried at 105° C. til the constant weight yielded >50% starting Asp; see run 24.
^{b}Due to the low solubility of Asp and/or Asp-hydrogensulfate in the system (1,2-PG − water), the reaction product was a suspension with predominant (>50%) undissolved Asp in the form of a white precipitate.
^{c}In this case, most of the Asp reacted yielding a solution of the product with only a minor amount of undissolved Asp which remained suspended in the reaction mixture. This suspended matter was removed during the filtration of the product. After drying at 105° C. the quantity of starting Asp was about 5%.
^{d}Since about 5% of starting Asp remained undissolved and removed by the final filtration of the product, the content of Asp in the final product (filtrate) was about 95% of starting Asp.
^{e}Due to the low solubility of Tyr and/or Tyr hydrogensulfate in the medium (1,2-PG + water), this experiment was performed at lower content of Si at 0.25% w/w; see run 8.
^{f}The reaction mixture was a suspension of a white precipitate in a viscous colourless supernatant. The undissolved fraction was removed during the final filtration of the product and dried at 105° C. til the constant weight. The amount of unreacted Tyr was 0.98 g or about 30% of the starting Tyr. Thus the stoichiometry in such a product is: n(Si):n(AA) ≈1:1.4.
^{g}This product also contains 2.86 g (2.86%) DL-malic acid from L-citrulline DL-malate (2:1) precursor.

### Example 20; Preparation of the Composition from the Present Invention with Analogues of Five Selected Amino Acids with 0.25% w/w Silicon (Si) and 50% w/w 1,2 Propylene Glycol

### Starting Materials (for 100 g Solution):

| | |
|---|---|
| (1) | m₁ g (n = 0.002225 mol; 0.25 mol. equiv. to Si) amino acid [L-threonine (Thr), |
| | L- L-tyrosine (Tyr), L-aspartic acid (Asp), L-proline (Pro), or beta-alanine |
| | (βAl)];^{a} |
| (2) | 50.00% w/w (40.00 g) 1,2-propylene glycol (3);^{a} |
| (3) | m₂ g purified water; |
| (4) | m₃ g sulfuric acid (H₂SO₄; 96% w/w);^{b} |
| (5) | m₄ g purified water; |
| (6) | 2.02% w/w (2.02 g) potassium silicate solution, type KASIL ®-6; contains |
| | 12.7% w/w K₂O and 26.5% w/w SiO₂; equivalent to: 0.2565 g |
| | K₂O, n(K₂O) = 0.002723 mol; 0.5353 g SiO₂, n(SiO₂) = 0.008909 |
| | mol; equivalent to 0.25% w/w Si in the final product.^{c} |

Total: 100% w/w (100.00 g)

The quantities of m₁-m₄ used in these experiments are as follows:

| Run | Amino acid component | m₁ [g]^{a} | m₂ [g] | m₃ [g]^{b} | m₄ [g] |
|---|---|---|---|---|---|
| 38 | Thr | 0.27 | 25.00 | 0.73 | 21.98 |
| 39 | Tyr | 0.40 | 25.00 | 0.73 | 21.85 |
| 40 | Asp | 0.30 | 25.00 | 0.73 | 21.95 |
| 41 | Pro | 0.26 | 25.00 | 0.73 | 21.99 |
| 42 | βAl | 0.20 | 25.00 | 0.73 | 22.05 |

^{a}The molar ratio of each amino acid (AA) to silicon was: n(Si):n(AA) = 1:0.25.
^{b}The quantity of sulfuric acid employed equals the sum of: (a) the amount of substance of K⁺ (from K₂O) and (b) 75% of the amount of substance of the corresponding amino acid (AA).
^{c} The quantity of potassium silicate solution (KASIL ® 6) corresponds to the content of silicon equal to 0.25% w/w Si in the final products.

Preparation: An amino acid (AA), according to column m₁ was added to 1,2-propylene glycol by stirring at r.t. during 5 min. Separately, sulfuric acid (m₃) was added dropwise to purified water (m₂) with stirring for 1 minute and thus obtained diluted sulfuric acid solution was added dropwise to the above-mentioned AA solution at 15-20° C. during 15 min. Separately, potassium silicate solution KASIL®-6 (2.02 g) was added to purified water (m₄) and homogenised by stirring for 5 min. Thus obtained diluted potassium silicate solution was added dropwise to the above-mentioned solution of amino acid (AA)-sulfuric acid in aqueous-1,2-propylene glycol solution at 13-17° C. during 25-30 min.

Thus obtained products, stabilised silicic acid solution according to the present invention, displayed the following key characteristics:

| | | Key characteristics of products from runs 38-42 of stoichiometry | | | | | |
|---|---|---|---|---|---|---|---|
| | | n(Si):n (AA) = 1:0.25, n/n | | | | | |
| | | | | | Density | Si | AA |
| | Amino Acid | | | pH at | [g/cm³] | content | content |
| Run | (AA) | Appearance | Odour | 22° C. | at 20° C. | [%, w/w] | [%, w/w] |
| 38 | Thr | A | N | 1.71 | 1.041 | 0.25 | 0.27 |
| 39 | Tyr^{a} | A | N | 1.66 | 1.038 | 0.25 | 0.40 |
| 40 | Asp^{a} | A | N | 1.68 | 1.038 | 0.25 | 0.30 |
| 41 | Pro | A | N / FO | 1.69 | 1.038 | 0.25 | 0.26 |
| 42 | βAl | A | N | 1.79 | 1.038 | 0.25 | 0.20 |

A = colourless, clear viscous liquid, approximate viscosity <1,2-propylene glycol (1,2-PG)
FO = faintly intensive, characteristic
N = odourless
^{a}No precipitation occurred upon standing of the final products at room temperature for several days. It seems that these products are stable against precipitation of the corresponding AAs and/or their hydrogensulfate salts (Asp, Tyr).

### Example 21: Preparation of the Composition from the Present Invention with Analogues of Five Selected Amino Acids 0.60% w/w Silicon (Si) and 1,2 Propylene Glycol

### Starting Materials (for 100 g Solution):

| | |
|---|---|
| (1) | m₁ g (n = 0.01068 mol; 0.50 mol. equiv. to Si) amino acid [L-leucine (Leu), L- |
| | lysine (Lys), L-aspartic acid (Asp), L-proline (Pro), and beta-alanine (βAl)]; |
| (2) | 40.00% w/w (40.00 g) 1,2-propylene glycol (3);^{a} |
| (3) | m₂ g purified water; |
| (4) | m₃ g sulfuric acid (H₂SO₄; 96% w/w);^{b} |
| (5) | m₄ g purified water; |
| (6) | 4.85% w/w (4.85 g) potassium silicate solution, type KASIL ®-6; contains |
| | 12.7% w/w K₂O and 26.5% w/w SiO₂; equivalent to: 0.616 g |
| | K₂O, n(K₂O) = 0.00654 mol; 1.28525 g SiO₂, n(SiO₂) = 0.02139 |
| | mol; equivalent to 0.60% w/w Si in the final product.^{c} |

Total: 100% w/w (100.00 g)

The quantities of m₁-m₄ used in these experiments are as follows:

| Run | Amino Acid (AA) | m₁ g^{a} | m₂ g | m₃ g^{b} | m₄ g |
|---|---|---|---|---|---|
| 43 | Leu | 1.40 | 25.00 | 2.15 | 26.60 |
| 44 | Lys•HCl | 1.95 | 25.00 | 2.15d | 26.05 |
| 45 | Asp | 1.42 | 25.00 | 2.15 | 26.58 |
| 46 | Pro | 1.23 | 25.00 | 2.15 | 26.77 |
| 47 | βAl | 0.95 | 25.00 | 2.15 | 27.05 |

^{a}The molar ratio of each amino acid to silicon was: n(Si):n(AA) = 1:0.50.
^{b}The quantity of sulfuric acid employed equals the sum of: (a) the amount of substance of K⁺ (from K²O) and (b) 75% of the amount of substance of the corresponding amino acid (AA) unless otherwise noted.
^{c} The quantity of potassium silicate solution (KASIL ® 6) corresponds to the content of silicon equal to 0.60% w/w Si in the final products.
^{d} L-Lysine is available as the hydrochloride salt. Therefore, the quantity of sulfuric acid, in this case, was equal to the sum of: (a) the amount of substance of K⁺ (from K₂O) and (b) 75% of the amount of substance of L-lysine (Lys). The other lysine amino group is already neutralized with an equimolar amount of hydrochloric acid (HCl) from starting, commercially available lysine hydrochloride (Lys•HCl).

Preparation: An amino acid (AA), according to column m₁ was added to 1,2-propylene glycol by stirring at r.t. during 5 min. Separately, sulfuric acid (m₃) was added dropwise to purified water (m₂) with stirring for 1 minute and thus obtained diluted sulfuric acid solution was added dropwise to the above-mentioned AA solution at 15-20° C. during 15 min. Separately, potassium silicate solution KASIL®-6 (4.85 g) was added to purified water (m₄) and homogenised by stirring for 5 min. Thus obtained diluted potassium silicate solution was added dropwise to the above-mentioned solution of amino acid (1)-sulfuric acid in aqueous-1,2-propylene glycol solution at 13-17° C. during 25-30 min.

Thus obtained products, stabilised silicic acid solution according to the present invention, displayed the following key characteristics:

| | | Key characteristics of products from runs 43-47 of stoichiometry | | | | | |
|---|---|---|---|---|---|---|---|
| | | n(Si):n (AA) = 1:0.5, n/n | | | | | |
| | | | | | Density | Si | AA |
| | Amino Acid | | | pH at | [g/cm³] | content | content |
| Run | (AA) | Appearance | Odour | 22° C. | at 20° C. | [%, w/w] | [%, w/w] |
| 43 | Leu | A | N / FO | 1.35 | 1.051 | 0.60 | 1.40 |
| 44 | Lys•HCl | A | N | 1.26 | 1.054 | 0.60 | 1.56 |
| 45 | Asp | A | N | 1.27 | 1.054 | 0.60 | 1.42 |
| 46 | Pro | A | N | 1.30 | 1.051 | 0.60 | 1.23 |
| 47 | βAla | A | N | 1.38 | 1.049 | 0.60 | 0.95 |

A = colourless, clear viscous liquid, approximate viscosity <1,2-propylene glycol (1,2-PG)
FO = faintly intensive, characteristic
N = odourless

### Example 22: Preparation of the Composition from the Present Invention Based on L-Tyrosine (Tyr) at 0.10% w/w Silicon (Si) and 0.05% w/w Silicon Content

(Run 48) Starting Materials for 100 g Solution of the Product with 0.10% w/w Si and 40% w/w 1,2-Propylene Glycol:

| | |
|---|---|
| (1) | 0.65% w/w (0.65 g; 0.003587 mol; 1 mol. equiv. to Si) L-tyrosine (Tyr);^{a} |
| (2) | 40.00% w/w (40.00 g) 1,2-propylene glycol (3); |
| (3) | 40.00% w/w (40.00g) purified water; |
| (4) | 0.30% w/w (0.30 g) sulfuric acid (H₂SO₄; 96% w/w);^{b} |
| (5) | 17.95% w/w (17.95 g) purified water; |
| (6) | 0.81% w/w (0.81 g) potassium silicate solution, type KASIL ®-6; contains |
| | 12.7% w/w K₂O and 26.5% w/w SiO₂; equivalent to: 0.10287 g |
| | K₂O, n(K₂O) = 0.001092 mol; 0.21465 g SiO₂, n(SiO₂) = |
| | 0.0035725 mol; equivalent to 0.10% w/w Si in the final product.^{c} |

Total: 100% w/w (100.00 g)

(Run 49) Starting Materials for 100 g Solution of the Product with 0.05% w/w Si and 5% w/w 1,2-Propylene Glycol:

| | |
|---|---|
| (1) | 0.33% w/w (0.33 g; 0.001821 mol; 1 mol. equiv. to Si) L-tyrosine (Tyr);^{a} |
| (2) | 5.00% w/w (5.00 g) 1,2-propylene glycol (3); |
| (3) | 85.00% w/w (85.00 g) purified water; |
| (4) | 0.30% w/w (0.30 g) sulfuric acid (H₂SO₄; 96% w/w);^{b} |
| (5) | 8.96% w/w (8.96 g) purified water; |
| (6) | 0.41% w/w (0.41 g) potassium silicate solution, type KASIL ®-6; contains |
| | 12.7% w/w K₂O and 26.5% w/w SiO₂; equivalent to: 0.05207 g |
| | K₂O, n(K₂O) = 0.000553 mol; 0.10865 g SiO₂, n(SiO₂) = |
| | 0.001808 mol; equivalent to 0.050% w/w Si in the final product.^{c} |

Total: 100% w/w (100.00 g)

Preparation: L-Tyrosine (0.30 g) was added to 1,2-propylene glycol and 85.00 g water by stirring at r.t. during 5 min. Sulfuric acid was added dropwise to the mixture with stirring for 1 minute and thus obtained reaction mixture was stirred at 15-20° C. for 15 min. Separately, to purified water (8.96 g), potassium silicate solution KASIL®-6 was added and homogenised by stirring for 5 min. Thus obtained diluted potassium silicate solution was added dropwise to the above-mentioned reaction mixture-sulfuric acid in aqueous-1,2-propylene glycol solution at 13-17° C. during 25-30 min.

Thus obtained product, stabilised silicic acid solution according to the present invention, displayed the following key characteristics:

| | | Key characteristics of products from runs 48-49 of stoichiometry | | | | | |
|---|---|---|---|---|---|---|---|
| | | n(Si):n (Tyr) = 1:1 | | | | | |
| | | | | | Density | Si | Tyr |
| | Amino Acid | | | pH at | [g/cm³] | content | content |
| Run | (AA) | Appearance | Odour | 22° C. | at 20° C. | [%, w/w] | [%, w/w] |
| 48 | Tyr^{e} | A | N | 1.54 | 1.032 | 0.10 | 0.60^{e} |
| 49 | Tyr^{f} | B | N | 1.69 | 1.002 | 0.05 | 0.33 |

A = colourless, clear viscous liquid, approximate viscosity <1,2-propylene glycol (1,2-PG)
B = colourless clear liquid
N = odourless
^{a} The molar ratio of silicon to L-tyrosine was: n(Si):n(Tyr) = 1:1.
^{b} The quantity of sulfuric acid employed equals the sum of: (a) the amount of substance of K⁺ (from K₂O) and (b) 100% of the amount of substance of Tyr.
^{c} The quantity of potassium silicate solution (KASIL ®-6) in run 48 corresponds to the content of silicon equal to 0.10% w/w Si in the final product.
^{d} The quantity of potassium silicate solution (KASIL ®-6) in run 49 corresponds to the content of silicon equal to 0.05% w/w Si in the final product.
^{e}The final product was a colourless, clear solution. However, upon standing the product at room temperature (20-23° C.) for a few days, slight precipitation occurred yielding about 50 mg of colourless crystalline precipitate / 50 g product (≈0.1%) or about 10% of starting Tyr.
^{f}The product was in the form of a colourless, clear solution. No precipitation occurred upon standing the product at room temperature (20-23° C.) for three days.

### Example 23. Preparation of the Composition from the Present Invention with 0.6% w/w Silicon (Si) Based on L-Alanine (Ala) and 1,2-Propylene Glycol (3)

### Starting Materials (for 100 g Solution):

| | |
|---|---|
| (1) | 1.91% w/w (1.91 g) L-alanine (Ala); n(Ala) = 0.02144 mol; |
| (2) | 40.00% w/w (40.00 g) 1,2-propylene glycol (3); |
| (3) | 49.71% w/w (49.71 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 3.53% w/w (3.53 g) sulfuric acid, 96%; 3.39 g H₂SO₄, n(H₂SO₄) = 0.03452 mol. |

Total: 100% w/w (100.00 g)

Preparation: L-Alanine (Ala) was dissolved in a mixture of purified water (20.00 g) and 1,2-propylene glycol by stirring at r.t. during 5 min. Separately, sulfuric acid was added dropwise to purified water (9.71 g) with external cooling at 15-20° C. for 5 min. and thus obtained diluted sulfuric acid solution was added dropwise to the above-mentioned L-alanine (Ala) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-alanine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=0.95/22° C.; the composition of the product is as follows:
(1) silicic acid; equivalent to 0.60% w/w of silicon (Si);
(2) 4.01% w/w L-alanine hydrogensulfate (1Ala);
(3) 0.00% w/w potassium L-alanine sulfate (2bAla);
(4) 1.78% w/w potassium hydrogensulfate (KHSO₄);
(5) 40.00% w/w 1,2-propylene glycol (3); and
(6) ad 100% w/w purified water.

### Example 24. Preparation of the Composition from the Present Invention with 0.6% w/w Silicon (Si) Based on L-Alanine (Ala) and Polyethylene Glycol 400 (4; PEG 400)

### Starting Materials (for 100 g Solution):

| | |
|---|---|
| (1) | 1.91% w/w (1.91 g) L-alanine (Ala); n(Ala) = 0.02144 mol; |
| (2) | 40.00% w/w (40.00 g) polyethylene glycol 400 (4; PEG 400); |
| (3) | 49.71% w/w (49.71 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 3.53% w/w (3.53 g) sulfuric acid, 96%; 3.39 g H₂SO₄, n(H₂SO₄) = 0.03455 mol. |

Total: 100% w/w (100.00 g)

Preparation: L-Alanine (Ala) was dissolved in a mixture of purified water (20.00 g) and polyethylene glycol 400 (4; PEG 400) by stirring at r.t. during 5 min. Separately, sulfuric acid was added dropwise to purified water (9.71 g) with external cooling at 15-20° C. for 5 min. and thus obtained diluted sulfuric acid solution was added dropwise to the above-mentioned L-alanine (Ala) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-alanine hydrogensulfate-sulfuric acid in aqueous-PEG solution at 15-20° C. during 20-30 min.

Thus obtained product, stabilised silicic acid solution according to the present invention, was in the form of an odourless, colourless, clear, slightly viscous solution; pH=1.10/20° C.; the composition of the product is as follows:
(1) silicic acid; equivalent to 0.60% w/w of silicon (Si);
(2) 4.01% w/w L-alanine hydrogensulfate (1Ala);
(3) 0.00% w/w potassium L-alanine sulfate (2bAla);
(4) 1.78% w/w potassium hydrogensulfate (KHSO₄);
(5) 40.00% w/w polyethylene glycol 400 (4; PEG 400); and
(6) ad 100% w/w purified water.

### Example 25. Preparation of the Composition from the Present Invention Based on L-Leucine (Leu) and 1,2-Propylene Glycol (3) with 0.6% w/w Silicon (Si)

### Starting Materials (for 100 g Solution):

| | |
|---|---|
| (1) | 2.81% w/w (2.81 g) L-leucine (Leu); n(Leu) = 0.02142 mol; |
| (2) | 40.00% w/w (40.00 g) 1,2-propylene glycol (3); |
| (3) | 49.36% w/w (49.36 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Leucine (Leu) was dissolved in a mixture of purified water (20.00 g) and 1,2-propylene glycol by stirring at r.t. during 5 min. Separately, sulfuric acid was added dropwise to purified water (9.36 g) with external cooling at 15-20° C. for 5 min. and thus obtained diluted sulfuric acid solution was added dropwise to the above-mentioned L-leucine (Leu) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-leucine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.24/20° C.; the composition of the product is:
(1) silicic acid; equivalent to 0.60% w/w of silicon (Si);
(2) 3.69% w/w L-leucine hydrogensulfate (1Leu);
(3) 1.43% w/w potassium L-leucine sulfate (2bLeu);
(4) 1.05% w/w potassium hydrogensulfate (KHSO₄);
(5) 40.00% w/w 1,2-propylene glycol (3); and
(6): ad 100% w/w purified water.

### Example 26. Preparation of the Composition from the Present Invention Based on L-Proline (Pro) and 1,2-Propylene Glycol (3) with 0.6% w/w Silicon (Si)

### Starting Materials (for 100 g Solution):

| | |
|---|---|
| (1) | 2.47% w/w (2.47 g) L-proline (Pro); n(Pro) = 0.02145 mol; |
| (2) | 40.00% w/w (40.00 g) 1,2-propylene glycol (3); |
| (3) | 49.70% w/w (49.70 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Proline (Pro) was dissolved in a mixture of purified water (20.00 g) and 1,2-propylene glycol by stirring at r.t. during 5 min. Separately, sulfuric acid was added dropwise to purified water (9.70 g) with external cooling at 15-20° C. for 5 min. and thus obtained diluted sulfuric acid solution was added dropwise to the above-mentioned L-proline (Pro) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-proline hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.10/20° C.; the composition of the product is:
(1) silicic acid; equivalent to 0.60% w/w of silicon (Si);
(2) 3.43% w/w L-proline hydrogensulfate (1Pro);
(3) 1.35% w/w potassium L-proline sulfate (2bPro);
(4) 1.05% w/w potassium hydrogensulfate (KHSO₄);
(5) 40.00% w/w 1,2-propylene glycol (3); and
(6) ad 100% w/w purified water.

### Example 27. Preparation of the Composition from the Present Invention Based on L-Serine (Ser) and 1,2-Propylene Glycol (3) with 0.6% w/w Silicon (Si)

### Starting Materials (for 100 g Solution):

| | |
|---|---|
| (1) | 2.25% w/w (2.25 g) L-serine (Ser); n(Ser) = 0.02141 mol; |
| (2) | 40.00% w/w (40.00 g) 1,2-propylene glycol (3); |
| (3) | 49.92% w/w (49.92 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Serine (Ser) was dissolved in a mixture of purified water (20.00 g) and 1,2-propylene glycol by stirring at r.t. during 5 min. Separately, sulfuric acid was added dropwise to purified water (9.92 g) with external cooling at 15-20° C. for 5 min. and thus obtained diluted sulfuric acid solution was added dropwise to the above-mentioned L-serine (Ser) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-serine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.15/20° C.; the composition of the product is:
(1) silicic acid; equivalent to 0.60% w/w of silicon (Si);
(2) 3.26% w/w L-serine hydrogensulfate (1Ser);
(3) 1.29% w/w potassium L-serine sulfate (2bSer);
(4) 1.05% w/w potassium hydrogensulfate (KHSO₄);
(5) 40.00% w/w 1,2-propylene glycol (3); and
(6) ad 100% w/w purified water.

### Example 28. Preparation of the Composition from the Present Invention Based on L-Glutamic Acid (Glu) and 1,2-Propylene Glycol (3) with 0.6% w/w Silicon (Si)

### Starting Materials (for 100 g Solution):

| | |
|---|---|
| (1) | 3.15% w/w (3.15 g) L-glutamic acid (Glu); n(Glu) = 0.02141 mol; |
| (2) | 40.00% w/w (40.00 g) 1,2-propylene glycol (3); |
| (3) | 49.02% w/w (49.02 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Glutamic acid (Glu) was dissolved in a mixture of purified water (20.00 g) and 1,2-propylene glycol by stirring at r.t. during 5 min. Separately, sulfuric acid was added dropwise to purified water (9.02 g) with external cooling at 15-20° C. for 5 min. and thus obtained diluted sulfuric acid solution was added dropwise to the above-mentioned L-glutamic acid (Glu) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-glutamic acid hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.20/20° C.; the composition of the product is:
(1) silicic acid; equivalent to 0.60% w/w of silicon (Si);
(2) 3.94% w/w L-glutamic acid hydrogensulfate (1Glu);
(3) 1.52% w/w potassium L-glutamic acid sulfate (2bGlu);
(4) 1.05% w/w potassium hydrogensulfate (KHSO₄);
(5) 40.00% w/w 1,2-propylene glycol (3); and
(6) ad 100% w/w purified water.

### Example 29. Preparation of the Composition from the Present Invention Based on L-Phenylalanine (Phe) and 1,2-Propylene Glycol (3) with 0.6% w/w Silicon (Si)

### Starting Materials (for 100 g Solution):

| | |
|---|---|
| (1) | 3.54% w/w (3.54 g) L-phenylalanine (Phe); n(Phe) = 0.02143 mol; |
| (2) | 40.00% w/w (40.00 g) 1,2-propylene glycol (3); |
| (3) | 48.63% w/w (48.63 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Phenylalanine (Phe) was dissolved in a mixture of purified water (20.00 g) and 1,2-propylene glycol by stirring at r.t. during 5 min. Separately, sulfuric acid was added dropwise to purified water (8.63 g) with external cooling at 15-20° C. for 5 min. and thus obtained diluted sulfuric acid solution was added dropwise to the above-mentioned L-phenylalanine (Phe) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-phenylalanine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.28/20° C.; the composition of the product is:
(1) silicic acid; equivalent to 0.60% w/w of silicon (Si);
(2) 4.23% w/w L-phenylalanine hydrogensulfate (1Phe);
(3) 1.61% w/w potassium L-phenylalanine sulfate (2bPhe);
(4) 1.05% w/w potassium hydrogensulfate (KHSO₄);
(5) 40.00% w/w 1,2-propylene glycol (3); and
(6) ad 100% w/w purified water.

### Example 30. Preparation of the Composition from the Present Invention Based on L-Tryptophan (Trp) and 1,2-Propylene Glycol (3) with 0.6% w/w Silicon (Si)

### Starting Materials (for 100 g Solution):

| | |
|---|---|
| (1) | 4.37% w/w (4.37 g) L-tryptophan (Trp); n(Trp) = 0.02140 mol; |
| (2) | 40.00% w/w (40.00 g) 1,2-propylene glycol (3); |
| (3) | 47.80% w/w (47.80 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Tryptophan (Trp) was dissolved in a mixture of purified water (20.00 g) and 1,2-propylene glycol by stirring at r.t. during 5 min. Separately, sulfuric acid was added dropwise to purified water (7.80 g) with external cooling at 15-20° C. for 5 min. and thus obtained diluted sulfuric acid solution was added dropwise to the above-mentioned L-tryptophan (Trp) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-tryptophan hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.28/20° C.; the composition of the product is:
(1) silicic acid; equivalent to 0.60% w/w of silicon (Si);
(2) 4.85% w/w L-tryptophan hydrogensulfate (1Trp);
(3) 1.82% w/w potassium L-tryptophan sulfate (2bTrp);
(4) 1.05% w/w potassium hydrogensulfate (KHSO₄);
(5) 40.00% w/w 1,2-propylene glycol (3); and
(6) ad 100% w/w purified water.

### Example 31. Preparation of the Composition from the Present Invention Based on L-Histidine (His) and 1,2-Propylene Glycol (3) with 0.6% w/w Silicon (Si)

### Starting Materials (for 100 g Solution):

| | |
|---|---|
| (1) | 3.32% w/w (3.32 g) L-histidine (His); n(His) = 0.02140 mol; |
| (2) | 40.00% w/w (40.00 g) 1,2-propylene glycol (3); |
| (3) | 46.66% w/w (46.66 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 5.17% w/w (5.17 g) sulfuric acid, 96%; 4.96 g H₂SO₄, n(H₂SO₄) = 0.0506 mol. |

Total: 100% w/w (100.00 g)

Preparation: L-Histidine (His) was dissolved in a mixture of purified water (15.00 g) and 1,2-propylene glycol by stirring at r.t. during 5 min. Separately, sulfuric acid was added dropwise to purified water (16.66 g) with external cooling at 15-20° C. for 5 min. and thus obtained diluted sulfuric acid solution was added dropwise to the above-mentioned L-histidine (His) solution at 15-20° C. during 5 min. Separately, potassium silicate solution KASIL®-6 was added to purified water (15.00 g) and shortly (1 min) homogenised by stirring. Thus obtained diluted potassium silicate solution was added dropwise to the above-mentioned solution of L-histidine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=0.81/20° C.; the composition of the product is:
(1) silicic acid; equivalent to 0.60% w/w of silicon (Si);
(2) 5.64% w/w L-histidine dihydrogensulfate [1His·H₂SO₄];
(3) 2.08% w/w potassium L-histidine sulfate hydrogensulfate (2bHis·H₂SO₄);
(4) 1.05% w/w potassium hydrogensulfate (KHSO₄);
(5) 40.00% w/w 1,2-propylene glycol (3); and
(6) ad 100% w/w purified water.

### Example 32. Preparation of the Composition from the Present Invention Based on L-Ornithine (Orn) and 1,2-Propylene Glycol (3) with 0.6% w/w Silicon (Si)

### Starting Materials (for 100 g Solution):

| | |
|---|---|
| (1) | 3.61% w/w (3.61 g) L-ornithine hydrochloride (Orn•HCl); n(Orn) = 0.02141 mol; |
| (2) | 40.00% w/w (40.00 g) 1,2-propylene glycol (3); |
| (3) | 48.56% w/w (48.56 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Ornithine hydrochloride (Orn·HCl) was dissolved in a mixture of purified water (20.00 g) and 1,2-propylene glycol by stirring at r.t. during 5 min. Separately, sulfuric acid was added dropwise to purified water (8.56 g) with external cooling at 15-20° C. for 5 min. and thus obtained diluted sulfuric acid solution was added dropwise to the above-mentioned L-ornithine (Orn) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-ornithine hydrochloride hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.12/20° C.; the composition of the product is:
(1) silicic acid; equivalent to 0.60% w/w of silicon (Si);
(2) 4.28% w/w L-ornithine hydrogensulfate hydrochloride (1Orn·HCl);
(3) 1.63% w/w potassium L-ornithine sulfate (2bOrn);
(4) 1.05% w/w potassium hydrogensulfate (KHSO₄);
(5) 40.00% w/w 1,2-propylene glycol (3); and
(6) ad 100% w/w purified water.

### Example 33. Preparation of the Composition from the Present Invention Based on L-Lysine (Lys) and 1,2-Propylene Glycol (3) with 0.6% w/w Silicon (Si)

### Starting Materials (for 100 g Solution):

| | |
|---|---|
| (1) | 3.92% w/w (3.92 g) L-lysine hydrochloride (Lys•HCl); n(Lys) = 0.02146 mol; |
| (2) | 40.00% w/w (40.00 g) 1,2-propylene glycol (3); |
| (3) | 48.25% w/w (48.25 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Lysine hydrochloride (Lys·HCl) was dissolved in a mixture of purified water (20.00 g) and 1,2-propylene glycol by stirring at r.t. during 5 min. Separately, sulfuric acid was added dropwise to purified water (8.25 g) with external cooling at 15-20° C. for 5 min. and thus obtained diluted sulfuric acid solution was added dropwise to the above-mentioned L-lysine (Lys) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-lysine hydrochloride hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.09/20° C.; the composition of the product is:
(1) silicic acid; equivalent to 0.60% w/w of silicon (Si);
(2) 4.52% w/w L-lysine hydrogensulfate hydrochloride (1Lys·HCl);
(3) 1.71% w/w potassium L-lysine sulfate (2bLys);
(4) 1.05% w/w potassium hydrogensulfate (KHSO₄);
(5) 40.00% w/w 1,2-propylene glycol (3); and
(6) ad 100% w/w purified water.

### Example 34. Preparation of the Composition from the Present Invention Based on Beta-Alanine (βAl) and 1,2-Propylene Glycol (3) with 0.6% w/w Silicon (Si)

### Starting Materials (for 100 g Solution):

| | |
|---|---|
| (1) | 1.91% w/w (1.91 g) beta-alanine (βAl); n(βAl) = 0.02144 mol; |
| (2) | 40.00% w/w (40.00 g) 1,2-propylene glycol (3); |
| (3) | 50.26% w/w (50.26 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: Beta-alanine (βAl) was dissolved in a mixture of purified water (20.00 g) and 1,2-propylene glycol by stirring at r.t. during 5 min. Separately, sulfuric acid was added dropwise to purified water (8.25 g) with external cooling at 15-20° C. for 5 min. and thus obtained diluted sulfuric acid solution was added dropwise to the above-mentioned beta-alanine (βAl) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of beta-alanine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.28/20° C.; the composition of the product is:
(1) silicic acid; equivalent to 0.60% w/w of silicon (Si);
(2) 3.01% w/w beta-alanine hydrogensulfate (1βAl);
(3) 1.21% w/w potassium beta-alanine sulfate (2bβAl);
(4) 1.05% w/w potassium hydrogensulfate (KHSO₄);
(5) 40.00% w/w 1,2-propylene glycol (3); and
(6) ad 100% w/w purified water.

### Example 35. Preparation of the Composition from the Present Invention Based on L-Methionine (Met) and 1,2-Propylene Glycol (3) with 0.6% w/w Silicon (Si)

### Starting Materials (for 100 g Solution):

| | |
|---|---|
| (1) | 3.20% w/w (3.20 g) L-methionine (Met); n(Met) = 0.02145 mol; |
| (2) | 40.00% w/w (40.00 g) 1,2-propylene glycol (3); |
| (3) | 48.97% w/w (48.97 g) purified water; |
| (4) | 4.85% w/w (4.85 g) potassium silicate solution, type KASIL ®-6; contains 12.7% |
| | w/w K₂O and 26.5% w/w SiO₂; equivalent to: 0.616 g K₂O, n(K₂O) = |
| | 0.00654 mol; 1.285 g SiO₂, n(SiO₂) = 0.02139 mol; |
| (5) | 2.98% w/w (2.98 g) sulfuric acid, 96%; 2.86 g H₂SO₄, n(H₂SO₄) = 0.02916 mol. |

Total: 100% w/w (100.00 g)

Preparation: L-Methionine (Met) was dissolved in a mixture of purified water (20.00 g) and 1,2-propylene glycol by stirring at r.t. during 5 min. Separately, sulfuric acid was added dropwise to purified water (8.97 g) with external cooling at 15-20° C. for 5 min. and thus obtained diluted sulfuric acid solution was added dropwise to the above-mentioned L-methionine (Met) solution at 15-20° C. during 5 min. Separately, potassium silicate solution KASIL®-6 was added to purified water (20.00 g) and shortly (1 min) homogenised by stirring. Thus obtained diluted potassium silicate solution was added dropwise to the above-mentioned solution of L-methionine hydrogensulfate-sulfuric acid in aqueous-1,2-propylene glycol solution at 15-20° C. during 20-30 min.

Thus obtained product, stabilised silicic acid solution according to the present invention, was in the form of an odourless, colourless, clear, slightly viscous solution; pH=1.22/20° C.; the composition of the product is:
(1) silicic acid; equivalent to 0.60% w/w of silicon (Si);
(2) 3.98% w/w L-methionine hydrogensulfate (1Met);
(3) 1.53% w/w potassium L-methionine sulfate (2bMet);
(4) 1.05% w/w potassium hydrogensulfate (KHSO₄);
(5) 40.00% w/w 1,2-propylene glycol (3); and
(6) ad 100% w/w purified water.

### Example 36. The Stability Study of the Composition from the Present Invention in Comparison to the Control Samples that could be Derived from the Prior Art

The six (6) control samples (from Examples 1-6) and two (2) compositions in accordance with the present invention (from Examples 7 and 8) were tested. All compositions had a concentration of stabilised silicic acid equivalent to the content of 0.6% w/w silicon (Si).

The stability study was performed in accordance with the guideline for the accelerated stability testing of pharmaceutical products, according to the International Conference on Harmonisation (ICH) Q1A (R2) (see: International Conference on harmonisation of technical requirements for registration of pharmaceuticals for human use: ICH Harmonised Tripartite Guideline: Stability Testing of New Drug Substances and Products Q1A (R2); Current Step 4 version, 6^{th} February (2003)) The study was conducted at accelerated stability testing conditions at 40-42° C./70% relative humidity (R.H.). The aim of the study was to determine the gelling time (t_{G}; expressed in [days]), which is the time period required for conversion of the freshly prepared liquid composition to become a clear-to-slightly opalescent semi-gel, which is no more liquid. T_{G} practically represents the shelf-life of the product.

From thus obtained t_{G} values for the tested and control samples, relative stability was calculated to numerically express the relative stability of the composition from the present invention against the stability of the control formulations. The relative stability is an empirical parameter obtained by a division of the gelling time (t_{G}) obtained with the composition from the present invention, products of Examples 7 and 8, and the result obtained by any of the control formulations. It represents a numerical value that describes how many times the formulation of the present invention is more stable than each control formulation derived from the prior art.

The results of this stability study are presented in the following Table.

The results of the stability testing^{a} of the composition from the present invention
in comparison to six (6) control formulations that could be hypothetically derived from
the combination of the closest prior art documents and the general knowledge.^{d}

| | | | |
|---|---|---|---|
| | | Stability (t_{G}; | |
| | | gelling time) at | Relative |
| No. | Sample | 40° C. [days]^{b} | stability^{c} |

A. Could be derived from the prior art:

| | | | |
|---|---|---|---|
| 1 | Glycine hydrochloride-glycerol based | 19 | >6.3x |
| | composition; see Example 1 | | |
| 2 | Glycine hydrochloride-sorbitol based | 4 | >30x |
| | composition; see Example 2 | | |
| 3 | Glycine dihydrogenphosphate-glycerol | 10 | >12x |
| | based composition; Example 3 | | |
| 4 | Glycine dihydrogenphosphate -sorbitol | 4 | >30x |
| | based composition; see Example 4 | | |
| 5 | Glycine dihydrogensulfate-glycerol based | 15 | >8x |
| | composition; see Example 5 | | |
| 6 | Glycine dihydrogensulfate-sorbitol based | 2 | >60x |
| | composition; see Example 6 | | |

B. The present invention:

| | | | |
|---|---|---|---|
| 7 | Product of Example 7 | >120 | 1 |
| 8 | Product of Example 8 | >120 | 1 |

^{a}The stability study was performed on the principles of the guideline for the accelerated stability testing of pharmaceutical products, according to the International Conference on Harmonisation (ICH) Q1A(R2); see literature reference 25.
^{b}Accelerated stability testing at 40-42° C./70% R.H. The gelling time (t_{G}; expressed in [days]) is a time period required for the conversion of the freshly prepared liquid composition to become a clear-to-slightly opalescent semi-gel, which is no more liquid. T_{G} practically represents the shelf-life of the product.
^{c}Relative stability is an empirical parameter obtained by a division of the gelling time obtained with the composition from the present invention, products of Examples 7 and 8, and the result obtained by any of the control formulations. It represents a numerical value that describes how many times the formulation of the present invention is more stable than each control formulation derived from the prior art.
^{d}EP1487272B1; Choline-silicic acid complex with osmolytes and divalent trace elements; Inventor: D. A. R. Vanden Berghe; Applicant: Bio Minerals N.V. (BE); Priority date: 20 Mar. 2002.; U.S. Pat. No. 5,922,360A; Stabilized orthosilicic acid comprising preparation and biological preparation; Inventor: S. R. Bronder; Applicant: Bio Pharma Sciences B.V. (NL); Priority date: 7 Feb. 1994.

The results showed that the concept of “water-absorbing additive” or humectant-stabilising effect defined in the prior art document 17 is substantially not correct. Precisely, it is obvious that not all humectants exhibit stabilizing effects on ortho-silicic acid (OSA). For instance, samples based on sorbitol are relatively very unstable and the gelling occurred after only 2-4 days under accelerated testing conditions. Samples based on glycerol are far more stable, but still 6-12× less stable than the composition from the present invention.

The results showed that the composition of the present invention exhibits significantly higher stability against the polymerisation of contained ortho-silicic acid (H₄SiO₄), its oligomers, and lower, water-soluble polymers than any of the control formulations that could be possibly derived from the identified closest prior art

1. A stabilized silicic acid solution comprising:
(i) silicic acid, in the form of a mixture of:
ortho-silicic acid (H₄SiO₄), its
water-soluble oligomers, and
water-soluble polymers,
in a percentage equivalent to 0.01-1.00% w/w silicon (Si),
(ii) one or more amino acid hydrogensulfates 1AA, 1b, 1c, at a total amount of 0.015-21.53% w/w, 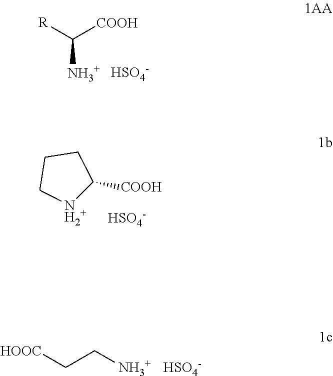
(iii) one or more sodium and/or potassium amino acid sulfates 2aAA, 2bAA, 2c, 2d, 2e, 2f, at a total amount of 0.00-24.24% w/w, 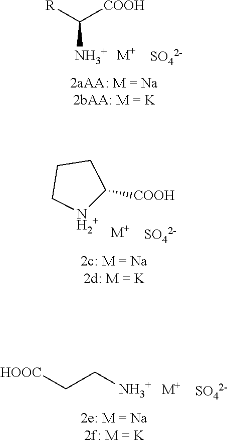
(iv) sodium and/or potassium hydrogensulfate (MHSO₄, where M=Na and/or K), at an amount of 0.00-9.70% w/w,
(v) a diol, selected from the group consisting of 1,2-propylene glycol (3), polyethylene glycol (PEG; 4), 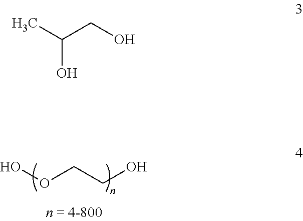
or their mixtures in a relative weight ratio:
$$w ( 3 ) : w ( 4 ) = 1. - 99. : 99. - 1. ,$$
in a percentage of 5-50% w/w within the composition, and
(vi) water, up to 100% w/w of the composition,
with the proviso that at least one of (iii) and (iv) is present.

2. A stabilized silicic acid solution according to claim 1, wherein the amino acids of 1AA, 2aAA, and/or 2bAA are selected from the group consisting of glycine (Gly), alanine (Ala), valine (Val), norvaline (Nva), leucine (Leu), isoleucine (Ile), norleucine (Nle), serine (Ser), threonine (Thr), aspartic acid (Asp), glutamic acid (Glu), phenylglycine (Phg), phenylalanine (Phe), tyrosine (Tyr), tryptophan (Trp), histidine (His), arginine (Arg), asparagine (Asn), glutamine (Gln), ornithine (Orn), lysine (Lys), methionine (Met), cysteine (Cys), citrulline (Cit), and salts thereof.

3. A stabilized silicic acid solution according to claim 1, wherein the term ‘water-soluble oligomer’ refers to linear chain, branched-chain, or cyclic oligomers formed by polycondensation of 2-12 ortho-silicic acid (OSA) molecules and the term ‘water-soluble polymer’ refers to polymers of general formula [SiOₓ(OH)₄₋₂ₓ]ₙ, wherein x=1-2, selected from the group consisting of:
sub-nano silicic acid (SNSA) of particles size 0.3-0.6 nm, wherein n=12-20 and M=700-1,400 [g/mol];
sub-nano (or subcolloidal) condensed silicic acid (SNCSA) of particles size 0.6-3 nm, wherein n=20-300 and M=1,400-2,000 [g/mol]; and,
larger SNCSA silicic acid of particles size 3-5 nm, wherein n=300-2,000 and M=20,000-140,000 [g/mol].

4. A stabilized silicic acid solution according to claim 1, wherein the water-soluble silicic acid oligomers and polymers are in the form of sub-colloidal particles having a size within the range of 0.1-10 nm

5. A stabilized silicic acid solution according to claim 1, wherein the term ‘water-soluble’ means that a clear water phase can be formed comprising said oligomers and/or polymers at a level of at least 10 ppm, preferably at least 100 ppm, more preferably at least 1000 ppm, at room temperature using plain water at neutral pH.

6. A stabilized silicic acid solution according to claim 1, comprising:
(i) silicic acid, in the form of a mixture of:
ortho-silicic acid (H₄SiO₄), its
water-soluble oligomers, and
water-soluble polymers,
in a percentage equivalent to 0.01-1.00% w/w silicon (Si),
(ii) glycine hydrogensulfate (1), 0.015-6.17% w/w, 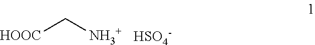
(iii) sodium and/or potassium glycine sulfate (2a,b) 0.00-5.64% w/w, 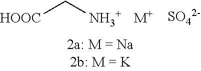
(iv) sodium and/or potassium hydrogensulfate (MHSO₄, where M=Na and/or K), 0.00-9.70% w/w,
(v) a diol, selected from the group consisting of 1,2-propylene glycol (3), polyethylene glycol (PEG; 4), 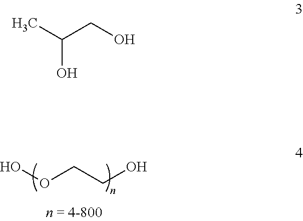
or their mixtures in a relative weight ratio:
$$w ( 3 ) : w ( 4 ) = 1. - 99. : 99. - 1. ,$$
in a percentage of 5-50% w/w within the composition, and
(vi) water, up to 100% w/w of the composition,
with the proviso that at least one of (iii) and (iv) is present.

7. A stabilized silicic acid solution according to claim 6, comprising:
(i) silicic acid, equivalent to 0.10-0.70% w/w silicon (Si),
(ii) glycine hydrogensulfate (1), 0.15-4.32% w/w,
(iii) sodium and/or potassium glycine sulfate (2), 0.00-3.95% w/w,
(iv) sodium and/or potassium hydrogensulfate (MHSO₄, where M=Na and/or K), 0.30-3.62% w/w,
(v) a diol, 10-45% w/w, and
(vi) water, up to 100% w/w of the composition.

8. A stabilized silicic acid solution according to claim 6, comprising:
(i) silicic acid, equivalent to 0.40-0.60% w/w silicon (Si),
(ii) glycine hydrogensulfate (1), 0.62-3.70% w/w,
(iii) sodium and/or potassium glycine sulfate (2), 0.00-3.38% w/w,
(iv) sodium and/or potassium hydrogensulfate (MHSO₄, where M=Na and/or K), 1.05-3.10% w/w,
(v) a diol, 30-45% w/w, and
(vi) water, up to 100% w/w of the composition.

9. A stabilized silicic acid solution according to claim 1 wherein polyethylene glycol (PEG; 4) is selected from the group consisting of: PEG 200, PEG 300, PEG 400, PEG 600, PEG 1000, PEG 1500, PEG 2000, PEG 3000, PEG 3350, PEG 4000, PEG 6000, PEG 8000, PEG 10000, PEG 12000, PEG 20000, PEG 35000 and mixtures of these substances.

10. A process for the preparation of a stabilized silicic acid solution, comprising the following steps:
A. dissolution of one or more amino acids in a mixture of a diol and purified water; which is realized by stirring at a temperature of 10-50° C. during 1-30 minutes,
B. separate stepwise addition of sulfuric acid (H₂SO₄) to one part of purified water at a temperature of 5-20° C. during 5-30 minutes,
C. addition of the sulfuric acid solution from step B into the solution from step A, at a temperature of 5-20° C. during 1-30 minutes,
D. separate preparation of sodium or potassium silicate solution in water either by:
the dissolution of solid substances in a part of purified water, or
dilution of liquid substances with a part of purified water, and
E. a dropwise addition of the solution from step D to a cooled solution from step C, at a temperature of 5-15° C. with an intensive stirring during 5-60 minutes, yielding the final liquid silicic acid composition.

11. A process according to claim 10, wherein the one or more amino acids are selected from the group consisting of glycine (Gly), alanine (Ala), valine (Val), norvaline (Nva), leucine (Leu), isoleucine (Ile), norleucine (Nle), serine (Ser), threonine (Thr), aspartic acid (Asp), glutamic acid (Glu), phenylglycine (Phg), phenylalanine (Phe), tyrosine (Tyr), tryptophan (Trp), histidine (His), arginine (Arg), asparagine (Asn), glutamine (Gln), ornithine (Orn), lysine (Lys), methionine (Met), cysteine (Cys), citrulline (Cit), proline (Pro), beta-alanine (βAl), and salts thereof.

12. A process according to claim 10 wherein:
(a) a molar ratio, n/n, of silicon (Si) to amino acid (AA) is:
$$n ( Si ) : n ( AA ) = 1 : 0.8 - 2.4 ,$$
(b) the amount of substance (n) of sulfuric acid equals the sum of the following amounts:
the amount of substance (n) of sodium and/or potassium (Na and/or K) from silicate; and
25-100% of the amount of substance (n) of the corresponding amino acid (AA):
$$n ( {H}_{2} {SO}_{4} ) = n ( M ) + 0.25 - 1. \cdot n ( AA ) ,$$
and,
(c) sodium and/or potassium silicate, xM₂O·ySiO₂, where M=Na and/or K, used in the process is:
in the solid form with 97-99% w/w sodium or potassium silicate, or
in the form of a liquid of a concentration of about 24-47% w/w sodium or potassium silicate,
which is characterized by the following molar ratio, x:y, n/n:x=1.00, y=1.00; x=1.00, y=1.60-1.70; x=1.00, y=2.00-2.10; x=1.00, y=3.30-3.40; x=1.00, y=3.90-4.00; or mixtures of these substances.

13. A process according to claim 12, wherein the one or more amino acids are or include histidine (His), arginine (Arg), ornithine (Orn), and/or lysine (Lys), wherein the side-moiety (R) is in the form of the free base, and the amount of substance (n) of sulfuric acid used in the process is increased for an equimolar amount of said employed AA, to ensure the conversion of the respective side-chain basic functional group into the corresponding hydrogensulfate salt.

14. A process for the preparation of a stabilized silicic acid solution according to claim 10, comprising the following steps:
A. dissolution of glycine (5) in a mixture of a diol and purified water; which is realized by stirring at a temperature of 10-50° C. during 1-30 minutes,
B. separate stepwise addition of sulfuric acid (H₂SO₄) to one part of purified water at a temperature of 5-20° C. during 5-30 minutes,
C. addition of the sulfuric acid solution from step B into the solution from step A, at a temperature of 5-20° C. during 1-30 minutes,
D. separate preparation of sodium or potassium silicate solution in water either by:
the dissolution of solid substances in a part of purified water, or
dilution of liquid substances with a part of purified water, and
E. a dropwise addition of the solution from step D to a cooled solution from step C, at a temperature of 5-15° C. with an intensive stirring during 5-60 minutes, yielding the final liquid silicic acid composition.

15. Stabilized silicic acid solution obtainable by the process as defined in claim 10.

16.-22. (canceled)
